SEQUENCE LISTING (13 sequences)

SEQ ID NO: 1 - DNA, 36 bp, Artificial
  Synthetic sequence
ataagaagcggccgcgagcggagcgggagcggatgc

SEQ ID NO: 2 - DNA, 33 bp, Artificial
  Synthetic sequence
tccatcggtcgacccaaggacttgtaggatcgc

SEQ ID NO: 3 - DNA, 33 bp, Artificial
  Synthetic sequence
gacgacgacaagatgcagcagccgcagccgcag

SEQ ID NO: 4 - DNA, 36 bp, Artificial
  Synthetic sequence
caggacagagcactagccatcttcctttgggggtgt

SEQ ID NO: 5 - DNA, 28 bp, artificial
  Synthetic sequence
caacatcgggtatgattccgtgatgttg

SEQ ID NO: 6 - DNA, 20 bp, Artificial
  Synthetic sequence
gagctgagctgcgagttcct

SEQ ID NO: 7 - DNA, 1463 bp, Musca musculus
gcggcggcggagcccggagcggaggccggaggctcccggcccgccggccccggagcggagcggagcggaggatgcagcagccgcagccgcaggggcagcagcagccggggccggggcagcaactgggggtccagggggcggcgccaggggccgggggcggcccgggagggggcccggggccagggccctgcctgaggcgggagctgaagctgctcgagtccatcttccaccgcggccacgagcgcttccgcattgccagcgcctgcctggacgagctgagctgcgagttcctgctggccggagccggaggggccggggccggggctgcgcccggaccccatctcccctcccggggatcggtgccgggggaccccgtccgcatccactgcaacatcacggaatcataccctgctgtgccccccatctggtcggtggagtccgatgaccctaacttggctgctgtcttggagaggctggtggacataaagaaagggaatactttgctcttgcagcatctgaagaggatcatctctgacctgtgtaaactctacaacctccctcagcatcctgatgtggaaatgctggatcagcccttgccagcagagcagtgcacgcaggaagaagtgtcttcagaagatgaagatgaagagatgcctgaggatacagaagacctagatcactatgaaatgaaagaggaagagccagccgagggcaagaagtctgaagatgatggcatcggaaaagagaacctggccatcctagagaaaattaaaaagaaccagaggcaagattacttaaatggtgcagtgtctggctcggtgcaggccactgaccggctgatgaaggagctcagggatatataccgatcacagagtttcaaaggcggaaactatgcagtcgaactcgtgaatgacagtctctatgactggaatgtcaaactcctcaaagttgaccaggatagcgctttgcacaatgatcttcagatcctgaaggagaaggaaggagcagacttcatcctacttaacttctcctttaaagataacttcccctttgacccaccgttcgtcagggttgtgtctccagtcctctctggagggtatgttctgggtggaggtgccatctgcatggaacttctcaccaagcagggctggagcagtgcctactccatagagtccgtgatcatgcagatcagcgccacgctggtgaaggggaaagcacgggtgcagtttggagccaacaaatctcagtatagcctgacgagagcacagcagtcctacaagtccttggtgcagatccatgaaaaaaacggctggtacacacccccaaaggaagatggctagccctggagtctcacccttcctcctccccaggcaccactggaccaattacctttgaatgctgtgtttggatctcacgctgcctctggtttccccccctcatttttcctggacgtgatagctctg

SEQ ID NO: 8 - DNA, 1435 bp, Homo sapiens
gcggcggcggcggagcccggagcgcaggccggaggctcccggcccgccggccccggagcggagcggagcggaggatgcagcagccgcagccgcaggggcagcagcagccggggccggggcagcagctggggggccagggggcggcgccgggggccgggggcggcccaggggggggcccggggccggggccctgcctgaggcgagagctgaagctgctcgagtccatcttccaccgcggccacgagcgcttccgcattgccagcgcctgcctggacgagctgagctgcgagttcctgctggctggggcaggaggggccggggcgggggccgcgcccggaccgcatctccccccacgggggtcggtgcctggggatcctgtccgcatccactgcaacatcacggagtcataccctgctgtgccccccatctggtcggtggagtctgatgaccctaacttggctgctgtcttggagaggctggtggacataaagaaagggaatactctgctattgcagcatctgaagaggatcatctccgacctgtgtaaactctataacctccctcagcatccagatgtggagatgctggatcaacccttgccagcagagcagtgcacacaggaagacgtgtcttcagaagatgaagatgaggagatgcctgaggacacagaagacttagatcactatgaaatgaaagaggaagagccagctgagggcaagaaatctgaagatgatggcattggaaaagaaaacttggccatcctagagaaaattaaaaagaaccagaggcaagattacttaaatggtgcagtgtctggctcggtgcaggccactgaccggctgatgaaggagctcagggatatataccgatcacagagtttcaaaggcggaaactatgcagtcgaactcgtgaatgacagtctgtatgattggaatgtcaaactcctcaaagttgaccaggacagcgctttgcacaacgatctccagatcctcaaagagaaagaaggagccgacttcattctacttaacttttcctttaaagataactttccctttgacccaccatttgtcagggttgtgtctccagtcctctctggagggtatgttctgggcggaggggccatctgcatggaacttctcaccaaacagggctggagcagtgcctactccatagagtcagtgatcatgcagatcagtgccacactggtgaaggggaaagcacgagtgcagtttggagccaacaaatctcaatacagtctgacaagagcacagcagtcctacaagtccttggtgcagatccacgaaaaaaacggctggtacacacccccaaaagaagacggctaaccctggagtatcacccttcctccctccccaggcaccactggaccaattacctttgaatgctgtatttggatctcacgctgcctctgtggttc

SEQ ID NO: 9 - protein, 295 aa, Caenorhabditis elegans
  misc_feature (1)..(411): Predicted sequence deduced from Caenorhabditis Elegans gene F25H2.8 (GenBank accession no. Z79754)
MACLKLKEIVLEKFPKNHRFQILSASVDELSKFINAEKGIIVANIEYPPPIWFESDDPVGMLQRLTTEESTNLQVRLVSDLCSYNLMPCQAPRDIDEGRGSDISDTTSEPIDDDMAGDEVDDDEEEEDDEAGDIEIVEAEEDTSDVGVSKEGLLKVSKIRQHLDGVGSATDRLMKERDIRSEFKNGIYELELYWIKLHKVDEDSPLDLKQDHLFFEKFPDPPFVRVVAPIGVLGGGAICMELLTKQGWSSAYSIESIQIAATLVKGARIFATSYSARAQQSFKSLQIHKGSTPKS

SEQ ID NO: 10 - protein, 236 aa, Drosophila melanogaster
  misc_feature (1)..(392): Predicted sequence deduced from Drosophila Melanogaster gene EG25E8 (GenBank accession no. AL009196)
MACLLKEIKLEKIFPKNHERFQILSVDELCFIGKHANITEYPSSPPWFESQLTQVLLCLNPPDLAPLTRGEEDSDEEEEGDESELALEKQRQDLGVSGSVQATDRLMKELRDIYRSFKYSELVNESYWNILVDDSPLHSDLQLKEKEGDILLNFKEPFPPFVRVVPISGGYVLGGAICMELLTKQGWSSAYEVIMQIAATLVKGKARIQFGAKQYSLARAQQSFKSLVQIHEKNGP

SEQ ID NO: 11 - protein, 295 aa, Saccharomyces cerevisiae; identical to SEQ ID NO: 9
  misc_feature (1)..(412): Predicted sequence deduced from yeast ubiquitin conjugating enzyme gene

SEQ ID NO: 12 - protein, 253 aa, Homo sapiens
MGLKEKLIFKNHERFISDELCFHNITESYPSSPIWFVSDDPLVLRLDTLQQKLDLCLYNLPDVELQPLTEEVSEEEEMDELEEEGSEDGKELAILEKKQRQDHLGAVSGSVQADRLMKELRDYRSSKGIYSVELNSLYDWKLHKVDDSPLHSDLQILKEKEGILLNFSFKFPFDPPFVRVVPVSGGYVLGGGACMELLTKQGWSSAYSIESVIMQIATLVKGKARQFGANKQYLARAQQSSVQIHEKNGWYPK

SEQ ID NO: 13 - protein, 294 aa, Musca musculus
MQQPQPQGQQQPGPGQQLGVQGAAPGAGGGPGGGPGPGPCLEKLEIFHERFISADELSCFAGHNITESYPPPIWVESDDPLVLRLDTLLQKRSDLCLYNLPDVELQPLEEVSDEEEMDELEEEGSEDGKELAILEKKQRQDLGAVSGSVQATDRLMKELRDIYRSSFKGYVELVNSLYDWNKLKVDDSLHDLQILKEKEGDILLNFSFKKPPPFVRVVPVSGGYVLGGGAICMELLTKQGWSSAYSIESVIMQIATLVKGKARQFGANKSQYSLRAQQSKSLVQIHEKNGWYPK

(19) United States
(12) Patent Application Publication
(10) Pub. No.: US 2011/0171681 A1
(43) Pub. Date: Jul. 14, 2011

(54) METHODS AND COMPOSITIONS FOR REGULATION OF STEM CELL SURVIVAL, PROLIFERATION, AND DIFFERENTIATION BY PROTEIN UBIQUITINATION

(75) Inventors: Yong-Jian GENG, Houston, TX (US); Michael WASSLER, Houston, TX (US)

(73) Assignee: BOARD OF REGENTS OF THE UNIVERSITY OF TEXAS SYSTEM, Austin, TX (US)

(21) Appl. No.: 13/070,124

(22) Filed: Mar. 23, 2011

Related U.S. Application Data
(63) Continuation of application No. 11/573,508, filed on Jul. 10, 2007, now abandoned.
(60) Provisional application No. 60/600,924, filed on Aug. 12, 2004.

(51) Int. Cl. C12Q 1/02; C12N 5/02

(52) U.S. Cl. 435/29; 435/375

(57) ABSTRACT

Compositions and methods for regulating in vitro cell growth are disclosed, and for providing undifferentiated stem cells or embryonic cells that are suitable for transplantation into damaged tissues or organs, or for use in tissue repair. A representative method includes causing the overexpression or underexpression of GalT binding protein (GtBP), also referred to as GalT associated protein (GTAP), in a cell such that ubiquitination of at least one cellular protein associated with cell adhesion and/or cell-to-cell interaction is correspondingly increased or decreased, causing inhibition of cell growth when GTAP is overexpressed and causing enhanced cell growth when GTAP is underexpressed by the cell. As a result, growth of the cell is altered or regulated.

### CROSS-REFERENCE TO RELATED APPLICATIONS

This application is continuation under 35 U.S.C. §120 of U.S. patent application Ser. No. 11/573,508 filed Feb. 9, 2007, which is the U.S. National Phase under 35 U.S.C. §371 of PCT Application No. PCT/US05/028823 filed Aug. 12, 2005, which claims the benefit under 35 U.S.C. §119(e) of U.S. Provisional Application No. 60/600,924 filed Aug. 12, 2004. The disclosures of those applications are hereby incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by grant number R01HL069509 awarded by the National Institutes of Health.

### BACKGROUND OF THE INVENTION

The development of a multicellular embryo from a fertilized egg lays the cornerstone for the birth of a new life. During the embryonic development, embryonic stem cells (ESCs) usually undergo a series of rapid, synchronous mitotic divisions and subsequently specialization or differentiation in function and morphology¹. The mitotic division and specialization of ESCs occur in a highly orchestral manner that requires a complex interplay between the endogenous genes and the microenvironmental factors. A differentiating ESC not only expresses numerous new proteins that governor cellular proliferation, adhesion, and signal transduction but also generates skeletal proteins and enzymes that metabolize macromolecules. On the other hand, almost all the organs and tissues in an adult contain certain numbers of multipotent, oligopotent or unipotent stem cells, namely a few such as the bone marrow stem cells, adipose tissue mesenchymal stem cells, neuron stem cells, endothelial cell progenitors, and myogenic stem cells². The adult stem cells behave in a manner similar to embryonic stem cells, capable of generating mature, functional cells, albeit having a relatively lower potency. Regulation of functional protein turnover inside or on the surface of stem cells represents a key event in determination of the survival, growth and differentiation of stem cells, regardless of their original tissues. Both embryonic and adult stem cells are candidate cells to be used for cellular therapy in regenerative medicine. In cardiology, stem cells are used to repair the myocardium with infarction³.

The ubiquitin-proteasome system plays a critical role in regulation of ATP-dependent protein degradation⁴. In physiology, ubiquitination enables a somatic cell to eliminate unwanted or degenerated proteins, thus maintaining homeostasis of proteins inside the cell. Accelerated or attenuated protein ubiquitination may alter a variety of cellular functions, including changing the rates of cell growth, survival, differentiation, as well as cell type switching. Many biological activities require appropriate ubiquitination of cellular proteins, such as the recycling of membrane receptors, endocytosis and fertilization.

Protein ubiquitination is usually achieved by the covalent binding of the 76 amino acid long, 8.5 kDa ubiquitin to the lysine residues of the target proteins⁵. This multi-step reaction catalyzed by a set of ubiquitin-carrying enzymes, termed ubiquitin-activating enzyme (E1), ubiquitin-conjugating enzyme (E2), and ubiquitin ligase (E3). First, E1 activates a single ubiquitin via a thiol ester bond. The activated ubiquitin is then transferred over to E2 enzymes that transiently carries the activated ubiquitin molecule as a thiol ester which together with ubiquitin ligases or E3s, targeting the substrate lysine residue. The major purpose of polyubiquitination is to target proteins for degradation through different proteasome complexes⁶. However, for receptors or other plasma membrane proteins that need to be regulated, the transfer of a single ubiquitin moiety serves as an internalization signal for transport of the protein to the lysosome with subsequent degradation⁷. Mono-ubiquitination may also influence protein transport and dislocation. Thus, protein ubiquitination may contribute to the lysomal protein degrading. In general, E1 and E3 enzymes exist in few isoforms, highly conservatively from yeast to man, whereas many different isoforms of E2 (ubiquitin-conjugating enzyme) can be found in a variety of cell lineages. The biological significance of such a variation in expression of the E2 isoenzymes in the cells is still unclear. Compared to limited numbers of the E1 and E3 isoenzymes, the E2 isoenzymes are more diverse and each of them may mediate ubiquitination in a cell type- or protein-selective fashion.

Oligosaccharides on cell surface proteins have been suggested to be involved in various cellular functions including cell-cell and cell-matrix interactions during embryogenesis^{8,9}. The involvement of Gal-containing complex N-glycans in cellular interaction during morula compaction and implantation has been suggested^{10,11}. Stage specific embryonic antigen-1 (SSEA-1) contains poly-N-acetyllactosamine structures and is specifically expressed on pre-implantation embryos and undifferentiated embryonic carcinoma cells. The oligosaccharide moieties of glycoconjugates in eukaryotic cells are synthesized by several different glycosyltransfeases and glycosidases. The first glycosyltransferase cloned and the most thoroughly studied was UDP-Gal:N-acetylglycosamin β1,4-Galactosyltransferase (GalT)¹². This enzyme is known to transfer Gal from UDP-Gal to a terminal N-acetylglucosamin (GcNAc) to from Gal β-4GlcNAc in the Golgi apparatus. The gene for GalT encodes two similar but not identical proteins due to differential transcription initiations¹²). Both proteins share a common catalytic domain but differ in their cytoplasmic domain; the short form has a cytoplasmic tail of only 11 amino acids, and the longer form an additional 13 amino acids giving rise to a 24-amino acid cytoplasmic domain^{13,14}. The shorter form resides mainly in the Golgi and a long form is located at the cell surface where it has a lectin-like binding property. The mechanism by which GalT elicits its function during development is currently not fully understood. In somatic cells, signaling from the glycosylating enzyme appears to result from protein interactions with its 24 amino acid cytoplasmic domain. This GalT I domain is associated with the actin cytoskeleton, and upon ligands binding to their surface receptors, it can trigger intracellular signal cascades. Clustering of surface GalT I with GlcNAc polymers or antibodies directed against this enzyme may also induce the subsequent tyrosine phosphorylation of focal adhesion kinase (FAK) and disorganization of actin stress fibers^{15,16}. Recently, results from GalT I null mice indicate that this glycosyltransferase may play a critical role in the regulation of proliferation and differentiation of embryonic cells¹⁷.

Cadherins are a group of multifunctional membrane proteins¹⁸, including epithelial (E)- and neural (N)-cadherins, which are major cell-cell adhesion receptors involved in the development, maintenance and function of most tissues. Cadherins also contribute to cell signaling, proliferation and differentiation. In cadherin-based adherens junctions (CAJs), the extracellular domains of transmembrane cadherins promote cell-cell adhesion by engaging in Ca²⁺-dependent homophilic interactions, while the cytoplasmic domains are linked to the actin cytoskeleton via α- and β-catenins¹⁹. Post-translational regulation of cadherin adhesive activities, including proteolytic processing of cadherins and disassembly of CAJs, plays crucial roles in rapid changes in cell adhesion²⁰, signaling and apoptosis²¹ but the molecular mechanisms involved in cadherin processing and CAJ disassembly remain mostly unknown. The embryonic stem (ES) cells from a null mutant mouse that lacks the cell adhesion molecule E-cadherin shows a defect in cell aggregation; which can be corrected by transfection with cDNA for either E-cadherin or N-cadherin driven by a constitutive promoter. While differentiating E-cadherin−/− ES cells are still able to express various early and late differentiation markers, they show a clear-cut deficiency in forming organized structures²². This phenotype can be rescued by constitutive expression of E-cadherin, which results exclusively in formation of epithelia. In contrast, rescue transfectants expressing N-cadherin show no epithelial structures, instead forming neuroepithelium and cartilage. Cadherins are also involved in embryogenesis²³, including striated muscle formation²⁴, nephrogenesis²⁵, the development at gastrulation²⁶ and formation of trophectoderm epithelium.²²

Closely related to plakoglobin (γ-catenin) in the armadillo family of proteins, β-Catenin²⁷ is located at the submembrane plaques of cell-cell adherens junctions where they form independent complexes with classical cadherins and α-catenin to establish the link with the actin cytoskeleton. Plakoglobin is also found in a complex with desmosomal cadherins and is involved in anchoring intermediate filaments to desmosomal plaques. In addition to their role in junctional assembly, β-catenin has been shown to play an essential role in signal transduction by the Wnt pathway that results in its translocation into the nucleus. Truncation of the tumour suppressor adenomatous polyposis coli (Apc) constitutively activates the Wnt/β-catenin signalling pathway²⁸. Apc has a role in development: for example, embryos of mice with truncated Apc do not complete gastrulation. Overexpression of Apc or Dickkopf 1 (Dkk1), a secreted Wnt inhibitor, blocks cushion formation. In wild-type hearts, nuclear β-catenin, the hallmark of activated canonical Wnt signalling, accumulates only in valve-forming cells, where it can activate a Tcf reporter. In mutant hearts, all cells display nuclear β-catenin and Tcf reporter activity, while valve markers are markedly upregulated. Concomitantly, proliferation and epithelial-mesenchymal transition, normally restricted to endocardial cushions, occur throughout the endocardium. There is a novel role for Wnt/beta-catenin signalling in determining endocardial cell fate. It has been reported²⁹ that Wnt/β-catenin signaling is activated at the inception of mammalian cardiac myogenesis and is indispensable for cardiac differentiation, at least in this pluripotent model system. The bicoid-related transcription factor Pitx2 is rapidly induced by the Wnt/Dv1/β-catenin pathway and is required for effective cell-type-specific proliferation by directly activating specific growth-regulating genes. Moreover, regulated exchange of HDAC1/β-catenin converts Pitx2 from repressor to activator, analogous to control of TCF/LEF1. Pitx2 then serves as a competence factor required for the temporally ordered and growth factor-dependent recruitment of a series of specific coactivator complexes that prove necessary for Cyclin D2 gene induction. The molecular strategy underlying interactions between the Wnt and growth factor-dependent signaling pathways in cardiac outflow tract and pituitary proliferation implies a similar mechanism for activation of cell-specific proliferation in other tissues³⁰. Beta-catenin plays a signal-integrating role in Wnt- and growth factor-dependent proliferation events in mammalian development by both derepressing several classes of repressors and by activating Pitx2, regulating the activity of several growth control genes.³¹

Progression through the cell cycle is catalyzed by cyclin-dependent kinases (CDKs) and negatively controlled by CDK inhibitors (CDIs)^{32,33}. Belonging to the p21CIP1/p27KIP1/p57KIP2 CDI family, p57 is an imprint protein containing four distinct domains following the heterogeneous amino-terminal region, which include, in order, a p21/p27-related CDK inhibitory domain, a proline-rich (28% proline) domain, an acidic (36% glutamic or aspartic acid) domain, and a carboxy-terminal nuclear targeting domain that contains a putative CDK phosphorylation site and has sequence similarity to p27 but not to p21³⁴. Most of the acidic domain consists of a novel, tandemly repeated 4-amino acid motif. p57 is a potent inhibitor of G1- and S-phase CDKs (cyclin E-cdk2, cyclin D2-cdk4, and cyclin A-cdk2) and, to lesser extent, of the mitotic cyclin B-Cdc2. In mammalian cells, p57 localizes to the nucleus, associates with G1 CDK components, and its overexpression causes a complete cell cycle arrest in G1 phase. In contrast to the widespread expression of p21 and p27 in human tissues, p57 is expressed in a tissue-specific manner, as a 1.5-kb species in placenta and at lower levels in various other tissues and a 7-kb mRNA species observed in skeletal muscle and heart. The expression pattern and unique domain structure of p57 suggest that this CDI may play a specialized role in cell cycle control. Repression of cyclin E-Cdk2-mediated phosphorylation of MyoD by p57(Kip2) may contribute to accumulation of MyoD at the onset of myoblast differentiation^{35,36}. p57(KIP2) is a cyclin-dependent kinase inhibitor and is required for normal mouse embryonic development. Mutations in CDKN1C or p57(kip2) have been identified in a small proportion of patients with Beckwith-Wiedemann syndrome, and removal of the gene from mice by targeted mutagenesis produces a phenotype with elements in common with this overgrowth syndrome³⁷.

A newly discovered protein family assigned to the epidermal differentiation complex (EDC) located on human chromosome 1q21. EDC comprises a large number of genes that are of crucial importance for the maturation of the human epidermis⁴⁴, and also in the progression of ailments such as breast cancer, tumorogenesis, inflammation and cardiomyopathy²⁰. So far, 27 genes of three related families encoding structural as well as regulatory proteins have been mapped. Recently, five new members (NICE-1, NICE-2, NICE-3, NICE-4 and NICE-5) of this complex were identified by subtractive hybridization technique on a keratinocyte cDNA library³⁸.

The yeast two-hybrid system³⁹ has been widely used to identify proteins that interact with other proteins in regulation of cell function. Many protein interactions with surface or intracellular receptors or enzymes appear to influence receptor signaling and functional regulation. There is great interest therefore in methods for the identification of novel or unanticipated receptor or enzyme-binding proteins. A proven method for identifying such protein interactions is the yeast two-hybrid screen, which involves screening the protein products of a cDNA library with a selected domain derived from a GPCR. Once it is established that a candidate protein produces a specific positive interaction within the yeast two-hybrid system, one will need to demonstrate further that this interaction is likely to occur in vivo⁴⁰. Co-immunoprecipitation, in which proteins of interest are co-purified with specific antibodies directed against the receptor or enzyme under study, can be used to address this important issue. In combination, the yeast two-hybrid screen and co-immunoprecipitation are a useful way to identify and sort through candidate ubiquitin-conjugating enzymes that interact intracellular or cell surface proteins prior to analysis in physiological studies⁴⁰.

### SUMMARY OF THE INVENTION

Methods and compositions are provided for protein ubiquitination in regulation of stem cell survival, growth and differentiation and applications for stem cell therapies and tissue repair. Highly activated ubiquitination occurs in undifferentiated, proliferating stem cells, which promote degradation of proteins that activate stem cells for differentiation. By controlling protein ubiquitination, the stem cell potency for growth and differentiation can be achieved. This process includes manipulation of the enzymes for ubiquitin synthesis, conjugation and ligation as well as the proteases for degradation of ubiquitinated proteins. Several key proteins targeted by ubiquitination in regulation of stem cell growth and adhesion and differentiation are described, which include, but are not limited to, those proteins involved in glycosylation (e.g., GalT), homeotypic adhesion (e.g., cadherins), intracelluloar signaling (e.g., catenins), and mitotic proliferation (e.g., cycline-kinase inhibitors). A unique ubiquitination pathway mediated by a GalT associated protein (GTAP), also referred to herein as GalT binding protein (GtBP), is presently disclosed, which may contribute to growth, adhesion, apoptosis and differentiation of embryonic and adult stem cells from various tissues. The protein ubiquitination system in stem cells of either embryonic or adult tissues, described herein, regulates the survival, growth, adhesion and differentiation of said stem cells. The ubiquitination system present in stem cells comprises evolutionarily conserved ubiquitin-carrying proteins referred to as ubiquitin-activating enzyme (E1), ubiquitin-conjugating (E2) and ubiquitin ligase (E3). The ubiquitination system comprises an isolated GalT associated protein (GTAP) that functions as an E2 enzyme, encoded by a cDNA sequence shown in FIG. 1, or encoded by a homolog of such cDNA from human fetal heart cDNA library (also shown in FIG. 1). The E2 enzyme GTAP of the ubiquitination system is structurally or functionally associated with NICE-5 or its homologs in the gene family epidermal differentiation complex. Regulation of GTAP expression and GTAP-mediated ubiquitination will alter stem cell maturation and cell lineage development, which is applicable to a variety of therapeutic applications.

Both murine and human GTAP cDNAs are cloned from respective embryonic libraries, showing a similarity to the epidermal differentiation complex (EDC), and is virtually identical to E2Q, one of the ubiquitin-conjugating enzymes (E2). It is demonstrated herein that GTAP exists abundantly in undifferentiating embryonic stem cell lines, embryonic tissue, and certain types of adult stem cells from the heart, blood vessels, adipose tissue as well as bone marrow. GTAP catalyzes ubiquitination of proteins involved in protein glycosylation, cell-cell or cell-matrix adhesion, cell cycle proceeding and apoptosis during early stages of embryonic development and certain diseases including cancer, heart failure, and neuron degeneration.

Accordingly, in certain embodiments of the present invention, a method for GTAP-mediated ubiquitination of proteins in stem cells or non-stem cells or cancer cells is provided. The method preferably comprises (a) causing ubiquitination of membrane proteins, such as growth factor receptors, glycosylating enzymes and adhesion proteins; (b) causing ubiquitination of signaling proteins, such as protein kinases, phosphorylating enzymes, the cadherin/Wnt/catenin complex, and transcription factors including NFκB and its inhibitor IκB; (c) causing ubiquitination of cell cycle regulating proteins, including cycline dependent kinases and their inhibitors, in particular p57(kip2), a nuclear protein encoded by an imprint gene; and thereby causing a controllable pattern of cell growth arrest or differentiation.

In accordance with certain embodiments of the present invention, a recombinant GalT associated protein (GTAP), sometimes also referred to herein as GalT binding protein (GtBP), is generated in mammalian cells or in bacteria by using a cDNA sequence shown in FIG. 1, or by using a human homolog of said cDNA with at least 95% sequence identity to a sequence shown in FIG. 1.

In accordance with certain embodiments of the invention, a method to deliver purified cDNA of GTAP or its analogs into stem cells by electroporation and liposome transfection is provided.

A method of regulating in vitro cell growth is provided according to another embodiment of the present invention. A representative method includes causing the overexpression or underexpression of GalT binding protein (GtBP), also referred to as GalT associated protein (GTAP), in the cell such that ubiquitination of at least one cellular protein associated with cell adhesion and/or cell-to-cell interaction is correspondingly increased or decreased, causing inhibition of cell growth when GTAP is overexpressed and causing enhanced cell growth when GTAP is underexpressed by the cell. In this manner, growth of the cell is altered or regulated as desired.

In some embodiments, the cell employed in the above-described method is an embryonic stem cell from embryonic tissues or an adult stem cell from adult tissue. In various embodiments, the method of regulating in vitro cell growth includes, increasing cell survival, enhancing cell migration, increasing the proliferation rate, promoting or deterring cell differentiation, or any combination of those results. In some embodiments, overexpression and activation of GTAP enhances ubiquitination of proteins and causes a decrease in cell adhesion and cell-cell interaction. In some embodiments, overexpression of GTAP correlates with a decrease in the amount of at least one cell surface protein chosen from the group consisting of GalT, cadherin, catenin and actin. Overexpression of GTAP correlates with an increase in the level of GTAP-mediated ubiquitination of GalT in said cell.

In some embodiments, overexpression of GTAP and other isoforms of ubiquitin-conjugating enzyme (E2) by cDNA transfection promotes ubiquitination of proteins that control the activity of cell cyclin-dependent protein kinase, including p21, p27 and p57(kip2) in stem cells, whereas underexpression by small double strand RNA interference (siRNA) suppresses protein ubiquitination of the cell cycle regulating proteins.

In accordance with certain embodiments of the invention, a method is provided for maintaining undifferentiated status of embryonic and adult stem cells which includes regulating protein ubiquitination through expression of the E2 enzymes such as GTAP and its analogs. In certain embodiments, maintaining growth and undifferentiated status of stem cells provides cells that are suitable for cell transplantation into damaged tissues or organs and for tissue repair.

In some embodiments of the present invention, a method is provided for controlling stem cell survival and cell lineage differentiation which includes regulating selective ubiquitination of key proteins for apoptosis, cross-membrane signal transduction, and cell-cell adherence, including the cadherin/Wnt/β-catenin system.

In some embodiments of the present invention, an in vitro method of altering survival, growth, adhesion or differentiation of a stem cell, a non-stem cell or a cancer cell is provided. This method comprises exposing the cell to a polypeptide inhibitor of GTAP mediated protein ubiquitination or a polynucleotide inhibitor of GTAP gene expression.

In accordance with certain embodiments of the present invention, a method of altering ubiquitination of at least one cellular protein associated with a cell function such as cell adhesion, migration, proliferation, differentiation or cell-to-cell interaction of a stem cell is provided. This method comprises one or more of the following steps: (a) increasing or decreasing expression of GTAP, or an analog thereof, by a cell, whereby GTAP or analog-mediated ubiquitination of said at least one protein is respectively increased or decreased; (b) activating or inactivating GTAP, or an analog thereof, by an agonist or antagonist, whereby GTAP or analog-mediated ubiquitination of said at least one protein is respectively increased or decreased; (c) causing changes in enzymatic reactions of GTAP, or an analog thereof, or another ubiquitin-conjugating enzyme (E2) in association with ubiquitin-activating enzyme (E1) and ubiquitin-ligase (E3) by modification of E1 and E3 enzyme expression and activities; and/or (d) stimulating or inhibiting degradation of ubiquitinated proteins by increasing or decreasing a 26S proteasome activity, whereby at least one cellular protein associated with cell adhesion, migration, proliferation, differentiation or cell-to-cell interaction is altered in the cell. In certain embodiments, in step (a) increasing or decreasing of GTAP comprises altering the levels of GTAP mRNA and proteins in the cell. In some embodiments, in step (b), activating or inactivating comprises administering to the cell an agonistic or antagonistic peptide or lipid whereby GTAP activities are altered or regulated. In some embodiments, step (c) comprises modification of the upstream (E1) or downstream (E3) portion of a GTAP enzymatic chain reaction, whereby ubiquitination of at least one protein is respectively decreased or increased. In some embodiments, in step (d), comprises increasing or decreasing 26S proteasome activity such that degradation of GTAP, or an analog thereof, or a ubiquitinated protein is inhibited or accelerated.

In accordance with certain embodiments of the present invention, a method of altering a cellular function in a stem cell comprises exposing the cell to a polypeptide inhibitor of GTAP mediated protein ubiquitination or a polynucleotide inhibitor of GTAP gene expression, whereby survival, growth, adhesion, differentiation or cell type switching of the stem cell is altered. In some embodiments, the analog comprises a dominant-negative polypeptide analog of GTAP which lacks the functional domain(s) or cofactor binding sites of GTAP. In some embodiments, the polynucleotide inhibitor comprises a small double-strand interference RNA targeting to GTAP mRNA.

In accordance with certain embodiments of the present invention, a method of indexing the pluripotency, multipotency, oligopotency or monopotency of a stem cell is provided which comprises assessing the level of polyubiquitination of the cell, and correlating the resulting level with pluripotency, multipotency, oligopotency or monopotency of the cell for growth, survival and differentiation into a cell type in the blood or somatic tissues or organs. In some embodiments, assessing the level of polyubiquitination comprises assessing the global polyubiquitination of proteins in pluripotent or multipotent embryonic stem cells. In some embodiments, assessing the level of polyubiquitination comprises selectively assessing GTAP-mediated polyubiquitination of a protein in the cell. In some embodiments, assessing the level of polyubiquitination comprises assessing GTAP protein and mRNA levels by an immunological, enzymatic or biochemical method, or a combination of any of those methods, in the cell. In certain of the above-described embodiments, the stem cell is an adult or embryonic stem cell, or is a cancer stem cell. These and other embodiments, features and advantages of the present invention will become apparent with reference to the following description and drawings.

### BRIEF DESCRIPTION OF THE DRAWINGS

FIG. 1. cDNA sequence comparison between mouse and human GTAP (also referred to as GtBP). The putative open reading frame (ORF) is depicted as boxed ATG and TAG, respectively.

FIG. 2. Northern blot of different mouse tissues or organs using a 500 bp 5′ cDNA probe of GTAP (GtBP). The amount of poly A RNA is normalized such that the β-actin hybridization signal is of comparable intensity in every lane (A). Quantitative RT-PCR of undifferentiated stem cells (ESC), differentiated embryonic bodies (EB at days 4-6) and adult heart (AH). The infold picture show FAM-related fluorescence of GAPDH (dotted lines) and GTAP (solid lines) for EB at day 4 and ESC (1, 4 and 3, 2) plotted against the number of PCR cycles.

FIG. 3. GTAP (GtBP) is evolutionarily conserved to proteins related to EDC and ubiquitin conjugating enzymes or analogs. Amino acid comparison of GTAP compared to sequences from C. Elegans, Drosophila Melanogaster, Yeast (Ubc 17), human GtBP (GTAP) and mouse GtBP (GTAP). The bold letters in black indicate the specific GalT binding amino terminal sequence. The domain homologous to the active site of ubiquitin conjugating enzymes (E2) is enclosed in a box.

FIG. 4. GTAP (GtBP) localizes to cytosol, cell membrane, nucleus and intracellular contacts during embryonic development. Antibodies was raised against the GalT binding amino terminal (N1) and the ubiquitin conjugating enzyme-like carboxy terminal region of GTAP (C3) (A). Immunofluoresence of NIH 3T3 fibroblast (B) and confocal microscopy of differentiated embryonic body stained with preimmun (i) or serum (N1)(ii) and visualized with goat anti rabbit antibodies conjugated to FITC. Western blot showing different protein level of GTAP during embryonic stem cell differentiation (D). Nuclear extraction of embryonic bodies (day 0-3): M; membrane and cytosolic fraction, Nu; nuclear fraction (E). Table diagram of GTAP protein level relative beta actin (F). Data are means from three separate experiments.

FIG. 5. GTAP (GtBP) co-localizes with cell surface GalT and attenuates cell spreading on laminin. 3T3 NIH fibroblasts overexpressing GFP-GTAP stained with preimmune serum (pi) or antibodies against GFP (i). White arrows indicate philopodia (A). Confocal microscopy of cells stained with antibodies against GalT and GFP. The asterix depicts the Golgi apparatus and the dotted circle, the nucleus (B). Lysate (L1) from 3T3 NIH fibroblasts overexpressing a truncated form of GalT fused to GFP (TL/GFP), were immunoprecipitated using antibodies to GTAP (C3) and analyzed with western blot using antibodies against GFP (C). Lysate (L2) from 3T3 cells that overexpress GTAP (GTAP/GFP) were subjected to immunoprecipitation with antibodies against the catalytic domain of cell surface GalT and subsequently analyzed with western blot using antibodies against GFP (D). Cells overexpressing GFP fusion protein of GTAP plated on Fibronectin (FN), Laminin (LM) and Mock transfected cells (Con) plated on Laminin (F).

FIG. 6. GTAP (GtBP) regulates embryonic body formation and cell growth. A colony of undifferentiated cells expressing GTAP fused to hemaglutinin (GTAP-HA) was fixed and stained with antibodies against GalT (A). Lysates from mock and GTAP cDNA transfected embryonic stem cells (ESC-GTAP) subjected to cell surface biotinylation, were immunoprecipitated using antibodies against GalT and hemaglutinin (HA). Biotinylated proteins were analyzed using streptavidin conjugated to HRP (B). Growth curve and BrD staining of ESC transfected with plasmid only (pIRES) and with GTAP-HA cDNA (GTAP) (C-D). Embryonic bodies formed from cells containing plasmid only (Mock), cells ectopically expressed GTAP (GTAP-HA) and siRNA knock-out cells (GTAP/siRNA) isolated from different time points (1-4 days post differentiation) (E). Table diagram showing the diameter of embryonic bodies formed from stable cell lines (F). Data are means from three separate experiments.

FIG. 7. GTAP (GtBP) regulates the protein level of GalT and cadherin/catenin. Cells containing plasmid only (Mock), cells transfected with cDNA of GTAP (GTAP-HA) were subjected to RT-PCR using primers to GTAP, GalT, E-cadherin and GAPDH (see Materials and Methods) (A). Cell lysates from cell lines stably expressing different amounts of GTAP-HA (#14-17) and knock-down GTAP (siRNA) were subjected to western blot and analyzed with antibodies against E-cadherin, p57, beta-catenin, GalT, actin and GAPDH (B). Confocal microscopy of mock transfected cells and cells overexpressing GTAP-HA using antibodies against actin and β-catenin (C). Confocal image of GTAP-HA expressing cells using antibodies against of cadherin and HA (D).

FIG. 8. GTAP (GtBP) is a ubiquitin conjugating enzyme that regulate ubiquitination of cell surface GalT and beta-catenin. His tagged GTAP was isolated using a Nickel agarose column, subjected to in vitro ubiquitination using biotinylated ubiquitin in the presence (lane 3-4)) and absence of ATP (lane 1-2). Samples were resuspended under non-reducing (NR) or reducing (R) condition, run on a 4-15% SDS-PAGE and finally blotted over to nitrocellulose. His tagged GTAP was detected with N1 antibodies (see Materials and Methods) and ubiquitination was determined with streptavidin conjugated to horse radish peroxidase (A). Lysates from cells treated with DMSO (−) or 5 μM MG132 (+) were subjected to immunoprecipitation with GalT antibodies and further analyzed for GalT (B) and ubiquitinylated proteins using monoclonal antibodies against ubiquitin (C). Biotinylated cell surface proteins from GTAP cDNA transfected embryonic stem cells (GTAP-HA) were subjected to immunoprecipitation using antibodies against hemaglutinin (HA) and biotinylated proteins were detected using strepavidin conjugated to HRP (D). Lysates from cells treated with DMSO (−) or 5 μM MG132 (+) were subjected to immunoprecipitation with antibodies against catenin (E) western blot using monoclonal antibodies against ubiquitin (F).

FIG. 9. GTAP (GtBP) regulates ubiquitination of the cell cycle inhibitor p57(kip2) and its transport to the nucleus. In vitro ubiquitination of p57 was done using biotinylated ubiquitin. Ubiquitinylated proteins and p57 were detected with streptavidin conjugated to horse radish peroxidase (HRP) and monoclonal antibodies to p57, respectively (A-B). Lysates from stably transfected cells treated with DMSO (−) or 5 μM MG132 (+) were subjected to immunoprecipitation using p57 polyclonal antibodies and western blot using antibodies to ubiquitin (C). Confocal image showing mock transfected cells (Mock) and cell containing GTAP cDNA (GTAP-HA) stained with p57 antibodies (D). Nuclear extracts of mock and GTAP transfected cells were subjected to immunoblotting and stained for GTAP and p57: M; membrane and cytosol, Nu; nucleus (F).

### DETAILED DESCRIPTION

Overview

Proliferation and differentiation of stem cells, including embryonic and adult stem cells, are regulated by a broad range of genes important for cellular metabolism, migration, adhesion, cell-cell interaction, signal transduction and cell cycle regulation. We demonstrated that the global levels of protein ubiquitination in undifferentiated stem cells are much greater than that in differentiated cells or mature tissues. Selective ubiquitination of certain proteins by manipulation of certain enzymes responsible for ubiquitin synthesis, activation, conjugation or ligation could influence the potency of stem cell growth and differentiation. Galactosyltransferase I (GalT) is a type II transmembrane glycoprotein that has been implicated in several important cellular processes, e.g., as a receptor during laminin-dependent cell migration, metastasis, reproduction and development. To search for putative interacting and signaling partners to GalT in development, we screened an embryonic mouse cDNA library using the cytoplasmic domain of GalT in a yeast two hybrid approach. A GalT associated protein (GTAP) cDNA was cloned and characterized from both murine and human embryonic libraries. Northern blot revealed that GTAP was highly expressed in testis and ovary and medium- to low-expression in kidney, lung, thymus and heart as a 1.6 kb message. The protein translated from the predicted open reading frame (ORF) of GTAP show 50-70% similarity to NICE 5, a recently discovered gene family with unknown function, located in the epidermal differentiation complex (EDC). This complex is located on human chromosome 1q21 and is comprised of a large number of evolutionarily conserved genes from C. Elegans to man, important in signal transduction as well as in the structural properties of epidermis. Furthermore, the carboxy terminal end of GTAP shows sequence similarities to ubiquitin conjugating enzyme E2 (Ub-E2) implicated in a variety of cellular functions. Immunoprecipitation and Western blot using antibodies made against HIS and GST fusion proteins of GTAP, identified a protein of 52-55 kDa in both 3T3 fibroblasts, testis and in embryonic stem cells (ESC). Transfection of 3T3 fibroblasts with a cDNA encoding a fusion protein of GTAP and a green fluorescent protein (GTAP-GFP) showed localization to the cytoplasm, philopodia and lamellipodia as well as the nucleus. Quantitative RT-PCR analysis demonstrated that the level of GTAP mRNA was initially high in undifferentiated cells but dramatically decreased during embryonic body formation. Immunohistochemical staining showed GTAP staining at intracellular contacts in differentiating embryonic bodies (dEBs). Overexpression of GTAP fused to hemaglutinin in mouse embryonic fibroblasts severely attenuated cell spreading on laminin and the formation and growth of embryonic bodies. In further studies, we showed that GalT, cadherin and catenin were subjected to ubiquitination in a GTAP-proteosome dependent manner. Overexpression of the ubiquitin-conjugating enzyme with subsequent decrease in their protein level. In still further studies, the cycline-dependent kinase inhibitor p57(kip2), a cell cycle inhibitor, was subjected to ubiquitination. As described in more details below, by cDNA cloning and characterization of ubiquitin-conjugating enzyme, GTAP, a putative new member of the EDC family, we have shown that GTAP-mediated ubiquitination of intracellular proteins may play a role in regulation of cell migration, growth and proliferation.

Materials and Methods

Materials. Swiss 3T3 were purchased from ATCC (Bethesda, Md.), and plated on plastic tissue culture dishes (Corning) and maintained in Dulbeccos Modified Essential Medium (GIBCO BRL,) supplemented with 10% BCS at 37° C. in 5% CO₂ and 800 μg/ml Geneticin (G418), when indicated. ESC were propagated and maintained in DMEM containing high glucose, nonessential amino acids, 200 mM L-glutamine, 100 μM MTG, 20% fetal calf serum (FCS) and 1000 U/ml of leukemia inhibitor factor (LIF) unless otherwise indicated. Rabbit polyclonal antibodies and mouse monoclonal antibodies to GFP were purchased from Clontech (San Diego, Calif.). GST antibodies were purchased from Chemicon International (Temecula, Calif.). Antibodies were made against the catalytic domain of recombinant of murine GalT as described earlier⁴¹. Antibodies against E-cadherin were from BD Bioscience (Palo Alto, Calif.) and polyclonal antibodies against Kip2 p57 from Sigma Aldrich. Monoclonal anti-p57kip2 antibody (clone KP39 from Sigma, product no. P2735) (1:4000). Antibodies against beta actin and GAPDH were purchased from Sigma. Mouse ESC was purchased from Stem Cell Technology (Vancouver, Canada). Horseradish peroxidase secondary antibodies were used (Santa Cruz Biotechnologies, Inc., Santa Cruz, Calif.), unless otherwise stated. All vectors were purchased from Clonetech (La Jolla, Calif.) and all chemicals were from Sigma (St. Louis, Mo.) unless stated otherwise.

Construction of the GAL4-GalT cytoplasmic domain two hybrid vector. A yeast two hybrid DNA binding (DB) domain with the cytoplasmic domain of GalT located upstream of the bulky GAL4 DB domain^{15,42,43}. A 75 bp oligomer encoding the N-terminal portion (aminoacids 1-24) of GalT was ligated into the BamHI and Nco I site of a modified GAL4 DB plasmid (D151, kindly given by Rob Brazas, University of California at San Francisco, Calif.). A mouse embryonic library in phage (X act), kindly given by Eric Olsen, UT MD Anderson Cancer Center) was automatically subcloned into a plasmid library using bacterial strain RB4E, kindly given by the Steve Elledge lab. As controls for putative interacting clones, a GAL 4 activation domain (AD) fusion proteins containing Raf, E12 or SNF 1 (kindly given by Stevan Marcus, UT MD Anderson Cancer Center, UT-Houston, Tex.) were used.

Two Hybrid Screening

GT-D151 was screened against an oligo dT and random primed 10-day old mouse embryonic cDNA in a pACT vector (Clontech, La Jolla, Calif.). Transformation of GT-D151 and library was done by modification of the method reported previously¹⁶. Briefly, Yeast strain HF7C (MATa ura3-52, his3-200, ade2-101, lys2-801, trp1-901, leu2-3, 112, gal4-542, gal80-538, LYS2::GAL1, GAL1-HIS3, URA3:: (GAL4 17-mers)₃-CYC1-LaZ) were grown in 100 ml of YPD to an OD₆₀₀ of 0.5-0.7 and harvested by centrifugation and resuspended in 50 ml of sterile water and centrifugated again. The washed cells were rewashed with 20 ml LiTE (100 mM LiOAc, 10 mM Tris-HCl, pH 8.0, 1 mM EDTA) and resuspended in 8 ml of LiTE. After a brief incubation (10 min) at room temperature, the cell suspension was mixed with a 10 mg of denatured salmon sperm carrier DNA, 150 μg GT-D 151 and 200 ug of mouse embryonic library cDNA described earlier was added. After incubation in 30° C. for 10 min, sterile LiPEG (40% PEG 3350, 1.0 M LiOAc, 1×TE. pH 8.0) was added and mixed. The cell suspension was incubated in a 500 ml flask at 30° C., 30 min, while shaking at 200 rpm. DMSO was added to a final concentration of 10% (v/v) and the cell suspension was incubated at 42° C. for 15 min, chilled on ice, and the cells were resuspended in 1×TE buffer. An aliquot of 200 ml of the suspension was plated on 15-cm drop-out agar plates (SC-trp, leu, his) containing 5 mM 3-AT. Protein interactions were identified using a modified β-galactosidase filter assay (Clontech, CA). His⁺ colonies were transferred to nitrocellulose membrane, permeabilized in liquid nitrogen, and placed on Whatman No. 3 filter paper soaked in Z-buffer (60 mM Na₂HPO₄, 40 mM NaH₂PO₄, 10 mM MgCl₂, 50 mM β-mercaptoethanol) containing 1.0 mg/ml X-gal (Gibco BRL, MD). Colonies that turned blue after 1-5 h were screened for interaction again as described above. To identify positive clones that did not activate the lacZ gene, colonies were repetitively replica plated on drop-out agar plates (-Leu) and screened for loss of bait. Specific clones were harvested in drop-out media (-Leu, -His) and the GAL4AD plasmid cDNA (prey) was isolated by electroporation and amplification in E. Coli. Prey plasmids were re-transformed into yeast together with GT-D151 or GAL4 DB fusion plasmids containing Raf, E12 or SNF 1 and tested for specificity. Sequencing was performed using an ABI Fluorescent Sequencer and searched for homology using a NCBI BLAST search program.

cDNA Cloning of GTAP

In order to find a full length clone of GTAP, a cDNA clone isolated from the two hybrid screen (26.11a), was labeled with α-P³² using nick translation. Approximately 5×10⁴ recombinants were screened using a λgt11 cDNA library (10 day old mouse embryo) by plaques hybridization. After three cycles of plaque purification, several clones were isolated and subcloned into pBluescript KS⁻ vector using Escherichia coli K-12 strain XL-1 Blue. The nucleotide sequences of the inserts were then determined using the Thermo Sequenase Cycle Sequencing Kit (Amersham Pharmacia Biotech UK) with M13 universal and reverse primers. Two overlapping clones, m04 and m13, respectively were put together using laser gene Megaline software resulting in 1.5 kb long GTAP cDNA.

Northern Blot

Commercially available Nylon membrane (BD Bioscience, Palo Alto, Calif.) containing 10 μg of total poly A⁺ RNAs from mouse organs or tissues were hybridized with an [α-³²P] dCTP-labeled 500-bp Bgl II-Bgl II fragment from GTAP cDNA. Stringency washes (65° C.) were 1×10 min with 2×SSC, 0.1% SDS, and then 2×20 min with 0.5×SSC, 0.1% SDS.

Generation of Recombinant GTAP and GTAP-Fusion Proteins.

In order to localize GTAP in cells and determine if ectopic expression would impair any GalT specific function, we made fusion protein to GFP. Bluescript KS-containing GTAP were digested with Hind III and Bam HI. The fragment was gel purified and ligated into the multiple cloning site of pEGFP (Clontech, CA) downstream of GFP using the same restriction enzyme sites. Also, cDNA corresponding to the original clone (26.11a, 500 bp) isolated in the two-hybrid was isolated from pACT using Bgl II and gel purification (Quagen, CA). The fragment was subcloned into the multiple cloning site of pGEFP both down stream and upstream of GFP. E. Coli was transformed and propagated on LB plates containing 30 μg/ml kanamycin. The orientation of the inserts was determined by restriction endonuclease analysis. Finally, cDNA were transfected into 3T3 fibroblasts with Transfast™ reagent according to the manufacturer (Promega, Madison, Wis.). After incubation of cells in the presence of 800 μg/ml Geneticin, only clones that had a stable expression of GTAP-EGFP was used. To introduce GTAP into mouse embryonic stem cells, GTAP cDNA was subcloned into pIRES-hrGFP vector (Stratagene, CA) containing the human promoter for elongation factor 2 (EF-2) (kindly given by Dr Chung, Harvard Medical School, Belmont, Mass.) and with 3× hemaglutinin moieties down stream of the multiple cloning site. Briefly, GTAP was isolated from m04 KS vector (above) using PCR and Sal I/Not I containing primer pair; 5′-ATAAGAAGCGGCCG CGAGCGGAGCGGGAGCGGATGC-3′ (SEQ ID NO: 1) and primer 5′-TCCATCGGTCGACCCAAGG ACTTGTAGGATCGC-3′ (SEQ ID NO.: 2). The PCR fragment was digested with Not I and Sal I, run on a 1% TEA agarose gel and the resulting bands were cut out and purified using Qiagen PCR purification kit. The GTAP fragment was ligated into Sal/Not site of pIRES-hrGFP multiple cloning site and the resulting vector was electroporated into the bacteria DH5a. After selection on ampicillin containing LB agar plates the resulting clones were re-screened for GTAP using PCR with the same primers as above. Finally, neomycin resistance was created using recombination of a NEO cassette into the Cre/Lox site of pIRES (Stratagene), propagated in bacteria and selected using Kanamycin. Plasmids were then transfected into embryonic stem cells using electroporation. Clones stably expressing hemaglutinin tagged GTAP were selected and propagated for further use.

Construction of His-26.11a and GST-GTAP.

We chose to make antibodies to two different regions of GTAP. A His-tagged fusion protein was made against the amino acid terminal (N1) and a GST fusion protein to a region that excludes 26.11a (C3). 26.11a-His was made by digesting pACT-26.11a with Bgl II and the resulting fragment (500 bp) was cloned into the multiple cloning site of pTrcHis vector (Invitrogen, Carlsbad, Calif.). The orientation of the insert fragment was determined by restriction endonuclease analysis. Bacteria were transformed and colonies containing the cDNA were picked and grown to OD₆₀₀ of 0.6. The expression of the fusion protein was induced to by adding IPTG to a final concentration of 0.5 mM. After 4 hours at 37° C. the bacteria were spun down and the pellet were solubilized by sonication for 2×2 min in sarcosyl buffer (10 mM Tris-HCl, pH 8.0, 150 mM NaCl, 1.5% sarcosyl, 1 mM Mg, 20 mM imidazole, 5 mM β-mercaptoethanol, protein inhibitor cocktail. After centrifugation at 13,000×g, Triton X-100 was added to the supernatant to a final concentration of 3-4% (v/v) in order to block sarcosyl from interfering with the binding to the column. A volume of 5 ml of the supernatant was loaded onto a Ni-NTA column equilibrated in wash buffer (10 mM Tris-HCl, pH 8.0, 150 mM NaCl, 0.1% Triton X-100, 20 mM imidazole, 1 mM MgCl₂, 5 mM β-mercaptoethanol). After extensive washing, the fusion protein was eluted out with wash buffer containing 200 mM imidazole and run on a preparative 10% SDS -PAGE. After staining with Commassie, a 21 kDa band, corresponding to the His fusion protein was cut out, mixed with adjuvant and immunized into two rabbits.

GST-GTAP1S was constructed using ligation-independent cloning (LIC) of GTAP into pESP-2 (Stratagen, CA). Briefly, one insert specific sequences of GTAP were generated using PCR on the Blue script containing GTAP1. The upstream primers were designed with the vector specific 13 bp LIC specific sequence added to the 5′ end of GTAP1, 5′GTAP1S (5′GACGACGACAAGATGCAGCAGCCGCAGCCGCAG-3′) (SEQ ID NO: 3). The downstream primer 3′-GTAP contained the 12 bp specific vector LIC site and a stop codon (5′CAGGACAGAGCACTA GCCATCTTCCTTTGG GGGTGT-3′) (SEQ ID NO: 4). After treatment of the PCR product with Pfu DNA polymerase in the presence of dATP to generate 5′ single stranded overhang, the fragments were purified and cloned into pESP-2 vector. After transformation and amplification of cDNA into E. Coli, the insert was verified by PCR and sequencing. A fresh colony of Schizosaccharomyces Pombe was grown in EMM, and transformed according to the manufacturer. The transformants were then plated on EMM agar plates containing thiamine to select for colonies containing GST-GTAP cDNA. Positive colonies were picked and propagated in EMM/thiamine media until OD₆₀₀ of 0.2 was reached. After centrifugation at 1200×g for 5 min, the pellets were washed extensively in water and finally added to EMM media without thiamine to induce the expression of the fusion protein. GST-GTAP was extracted from the pellet using French press and finally purified using a GSH column. Antibodies were made in chicken from either native isolate or from nitrocellulose containing the protein. To exclude antibodies against GST, sera were run through GSH column (Stratagen, La Jolla, Calif.) and the run-through was saved for further analysis.

Western Blotting

Cells grown to 60-80% confluence and the cells were by scraped from the dishes. Approximately 4×10⁵ cells were washed twice with PBS (GIBCO BRL) and lysed in 1 ml of lysis buffer (10 mM Tris/HCl, pH 7.4, 150 mM NaCl, 0.5% NP40, 0.5% Triton X-100 and 1× protease inhibitors (Boehringer Mannheim, Germany). When indicated, 10 μM of MG132, a proteosome inhibitor, was included in the lysis buffer. After aspiration five times through a 25G needle, lysates were centrifuged at 13,000 rpm for 5 min and the pellet was discarded. Equal amounts of proteins (20 μg) were denatured in 2× Laemmli sample buffer containing 5% β-mercaptoethanol and loaded on 12% or 4-15% SDS-PAGE gels. After transfer to nitrocellulose membranes (Protran BA 85, Schleicher & Schuell) or PDVF (Immobilon P) the membranes were blocked with 5% dry milk or 5% BSA/5% normal goat serum (NGS) in TBS (10 mM Tris-HCl, pH 7.6, 150 mM NaCl, 0.5% Tween 20). After 1 hour at room temperature, the filters were washed and incubated with antibodies as described. Finally, goat anti mouse, goat anti rabbit IgG or Rabbit anti chicken IgY conjugated to horseradish peroxidase was added for an additional 45 min and the blots were developed using ECL (Amersham International).

Immunoprecipitation

Aliquots of various lysates were diluted two-fold with NET buffer (50 mM Tris-HCL, pH 7.5, 0.1% NP40, 0.25% [w/v] Gelatin, 150 mM NaCl) and incubated with 2.5 μl of polyclonal antibodies against GFP, GalT or 10 μl of GTAP antibodies (C3) for 4 h on a nutator at 4° C. A volume of 30 μl goat anti chicken IgY-agarose (Santa Cruz, Calif.) was added to the lysates and mixed for 1 h. The beads were pelleted briefly and washed three times with 500 μA wash buffer. The proteins were released from the beads by resuspension in 2× Laemmli buffer unless otherwise stated. After incubation at room temperature for 30 min the samples were subjected to Western blotting.

Indirect Immunofluorescence

Cells were grown to 80% confluence, dissociated and plated on cell culture-treated chamber glass slides (Nalge Nunc Intern, IL). After 24 h, cells were washed twice with PBS and immediately fixed with 4% paraformaldehyde/PBS for 30 min at room temperature. Cells were washed three times with PBS and permeabilized with 0.1% saponin/PBS for 15 min at room temperature and blocked with PBS/saponin/5% (NGS) for additional 20 min. Cells were treated with polyclonal antibodies against 26.11a (1:500), GFP (1:200) or GalT (1:500) 1 h at room temperature. After washing, cells were incubated with either goat anti-rabbit or anti-mouse IgG-biotin (1:200) for 45 min. Finally, streptavidin conjugated goat anti-rabbit IgG-FITC was added (1:400) and incubated 45 min at room temperature. Embryonic stem cells (ESC), embryonic bodies (EBs) and differentiated embryonic bodies (dEBs) were plated and propagated on glass chamber slides coated with laminin. The cells were fixed in 4% glutaraldehyde and washed in PBS. After blocking, cells were stained with GalT, endothelial cadherin (E-cad, 1:2500), β-catenin (1:500), β-actin (1:5000), GAPDH (1:500) for as above and finally viewed using Nikon Eclipse E800 microscope or confocal microscope.

Quantitative RT-PCR

Embryonic bodies (EBs) were prepared in hanging drops for 4 days, and then moved to 6-well plates coated with 0.1% gelatin to differentiate. Differentiated EBs (dEBs) were harvested 3, 6, 10, and 12 days after plating, lysed in CHAPS lysis buffer (50 mM 10 mM Tris, pH 8.5, 5 mM EDTA, 100 mM NaCl, 0.5% CHAPS, 2% Sodium Deoxycholate). Poly(A)+ RNA was extracted by using the Direct mRNA Purification Kit using magnetic porous glass (MPG: CPG Inc., Lincoln Park, N.J.). The isolated poly(A)⁺ RNA was reverse transcribed by using the SuperScript™ Preamplification System (Invitrogen, Carlsbad, Calif.). The resultant first-strand cDNA was subjected to quantitative real-time PCR. FAM-labeled LUX™ fluorogenic primes were designed by web-based software (http://www.invitrogen.com/). These sequence of GTAP: Labelled reverse primer: 5′CAACATCGGGTATGATTCCGTGATGTTG-3′ (SEQ ID NO.: 5), unlabelled forward primer: 5′-GAGCTGAGCTGCGAGTTCCT-3′ (SEQ ID NO.: 6). As a positive control and as a reference of initial amount of cDNA, we also amplified mRNA of glyceraldehyde 3-phosphate dehydrogenase (G-3-PDH). PCR was performed in a total volume of 50 μl of a buffer solution supplied by the Platinum Quantitative PCR SuperMix-UDG kit (Invitrogen) containing 1.5 unit of Platinum™ Taq polymerase. The thermal cycle protocol used was 95° C. for 30 sec, 60° C. for 1 min for 45 cycles with a programmable real-time thermal cycler (Rotor-Gene 3000: Corbett Research, Mortlake, Australia). Quantative analysis of data was performed using the Rotor-Gene software version 4. Experiments were repeated 3 times, and data were normalized by the amount of cDNA of a standard reference gene (G-3-PDH).

Ubiquitination of GTAP, GalT, E-Cadherin/β-Catenin and p57(kip2)

To verify that the carboxyl terminus of GTAP contain an active domain of ubiquitin conjugating enzymes, we analyzed thiolester formation to ubiquitin using an in vitro system. Aliquots containing 50 μg His tagged GTAP were bound to NTA beads column (Invitrogen). The beads were washed twice with reaction buffer (10 mM Hepes, pH 7.4, 5 mM MgAcetate, 150 mM creatin phosphate, 0.75 mg/ml creatin phophokinase) and resuspended in 25 μl of reaction buffer containing 1 mM DTT, 100 nM ubiquitin activating enzyme (E1) from rabbit, 5 μM ubiquitin, 5 μM biotinylated ubiquitin. The beads were then incubated in the presence or absence of 1 mM ATP at 30° C. for 90 min with occasional mixing. The beads were washed twice in reaction buffer; the beads were resuspended in 25 μl 2× thiol buffer (33 mM Tris/HCl, pH 6.8, 2.7 M urea, 2.7% SDS, 13% glycerol) or 2× reducing Laemmly buffer as earlier stated. After incubation at room temperature for 30 min the samples were loaded on 4-15% SDS PAGE gel and subjected to western blotting. Ubiquitinylated proteins were detected using streptavidin conjugated to horse radish peroxidase (SA-HRP) and compared proteins recognized by GTAP (N1) antibody. In order to see if GalT, E-cadherin, β-catenin and P57(kip2) could be ubiquinylated in vitro in a GTAP dependent way, lysates from undifferentiated stem cells were subjected to immunoprecipitation using antibodies to GalT, E-cadherin or beta catenin. Lysates were first precleared using 10 μl of protein A/G agarose (Santa Cruz) and subsequently mixed with 2.5 ul of GalT antibody, 10 μl of mouse anti E-cadherin or 5 ml of antibodies for p57. After over night incubation, beads were spun down and washed thoroughly in lysis buffer. The immunoprecipitates beads were then incubated and analyzed for ubiquitination as described above. In order to see if ubiquitination of these proteins was dependent on the proteosome pathway in vivo, mock transfected cells and cells ectopically expressing GTAP were incubated with DMSO or with 5 μM of the proteosome specific inhibitor MG132. After washing, the cells were scraped off in PBS and centrifuged. The pellets were lysed in RIPA buffer and subjected to immunoprecipitation. Precipitated proteins were transferred to nitrocellulose and analyzed with antibodies against ubiquitin (1:1000). After stripping the filter in Stripping buffer (Sigma-Aldrich) the filter was again blocked and analyzed for the amount of the respective protein.

Cell Surface Biotinylation

In order to label cell surface proteins, 3×10⁶ embryonic stem cells were seeded onto a E-well culture dish precoated with gelatin. At approximately 70% confluency the cells were washed three times with phosphate-buffered saline and then incubated with 0.5 ml of 0.6 mg/ml Sulfo-NHS-LC-biotin (Pierce) in phosphate-buffered saline (PBS) supplemented with 0.1 mM HEPES, pH 8.0 and 10 μM MG-132, a proteosome inhibitor. The media were withdrawn and the reaction was quenched by incubating the cells with PBS containing 0.5 ml of 50 mM ammonium chloride for an additional 10 min. The cells were then washed three times and incubated in solubilization buffer (0.5% Nonidet P-40, 0.5% TritonX-100, 1 mM phenylmethylsulfonyl fluoride, 1 mM 4-(2-aminoethyl)-benzenesulfonyl fluoride, 10 μg/ml leupeptin and 10 μM MG-132, in TBS) for 1 h at 4° C. Immunoprecipitation was carried out overnight at 4° C., using antibodies against GTAP, hemaglutinin or GalT. Antibodies were precipitated using 50 μl of protein A or G agarose (Santa Cruz). Immunoprecipitates were washed five times with solubilization buffer, resuspended in 25 μl protein sample buffer and samples run on 4-12% SDS-polyacrylamide gel electrophoresis. The filters were blocked overnight in TBS-T (20 mM Tris, pH 7.6, 145 mM NaCl, 0.1% Tween 20) containing 2% bovine serum albumin. After one hour incubation with streptavidin (1:40,000 dilution) coupled to horseradish peroxidase filters were washed extensively in TBS-T, and analyzed by enhanced chemiluminescence using an ECL kit (Amersham Pharmacia Biotech).

Nuclear Extraction

Aliquots of 1×10⁶ cells were collected by centrifugation and resuspended in cold PBS. The pellets were resuspended in 400 ul of buffer A containing 20 mM Hepes, pH 7.9, 10 mM KCl, 0.2 mM EDTA, and 0.25 mM PMSF. The cells were allowed to swell for 10 min on ice and 25 μl of 10% (v/v) of NP40 was added. After vortexing 10 seconds, the tubes were centrifuged and the supernatant saved (M). The pellets (nucleus) were resuspended in buffer B containing 20 mM Hepes, pH7.9, 0.4 M NaCl, 1 mM EDTA, 1 mM EGTA, 1 mM DTT and 0.25 mM PMSF. The samples were shaken in cold room for 15 min and centrifuged at max speed for 5 min. The supernatant (Nu) were saved for further study.

Antibodies for Western Blot Analysis and Immunocytochemistry

Antibodies against ubiquitin, GalT, GTAP, cadherins, catenines, p2′7, p57 and Cyclines, and markers for embryonic and adult stem cells were purchased or prepared by immunizing the peptides into the animals. Whole cell extracts for Western blot analysis were prepared by lysis and sonication (3×5 seconds) in RIPA buffer, and cell debris was removed by centrifugation at 13000 rpm for 20 min at 4° C. An equal volume of reducing 2× gel-loading buffer was added and the samples were boiled for 5 mins. Protein concentration in cell extracts was quantified with BCA protein assay kit (Pierce) using ELISA plate reader prior to addition of the loading buffer. Protein samples (20 μg) were electrophoretically separated on a 4-15% linear gradient SDS-polyacrylamide gel and electro blotted onto Nitrocellulose (BA85, Shleicher & Schull) or PVDF membrane (Protran). The filters were blocked for with TBS containing 3-5% milk and probed with antibodies.

Flow Cytometry

Stable cell lines were allowed to grow on gelatin coated cell culture dishes and then subjected to 10 μM BrDu (BD Bioscience, CA) for 3 hours at 37° C., CO₂. Cells were washed twice with PBS and harvested with trypsin. Aliquots of 1×10⁶ cells were pelleted and resuspended in 100 μl phosphate buffer saline (PBS) and subsequently fixed with 2 ml of −20° C. 70% (v/v) ethanol and incubated for 30 min at 0° C. After additions with 2 ml of 4 N HCl and centrifugation at 500×g for 5 minutes, the pellets were resuspended in 1 ml of 0.1 M tetraborate, pH 8.5. After centrifugation the pellets were mixed with 50 μl DPBS (PBS containing 0.5% Tween 3% Fetal Bovine Serum) and 30 μg DNAse and incubated for 1 h at 37° C. After centrifugation, the pellets were resuspended in DPBS containing antibodies against BrdU conjugated to FITC (1:50) After 20 minutes incubation in the dark at room temperature the cells were washed and finally resuspended in 1 ml of PBS containing 5 μg/ml of propidium iodide for nuclear staining.

Results

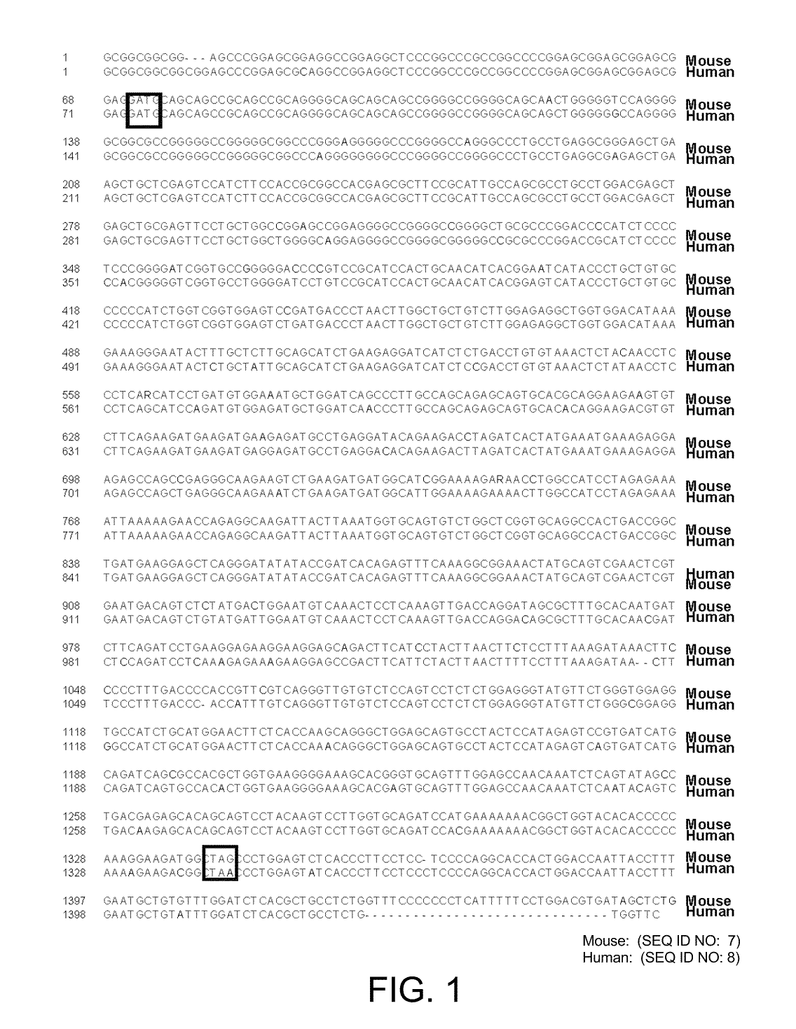

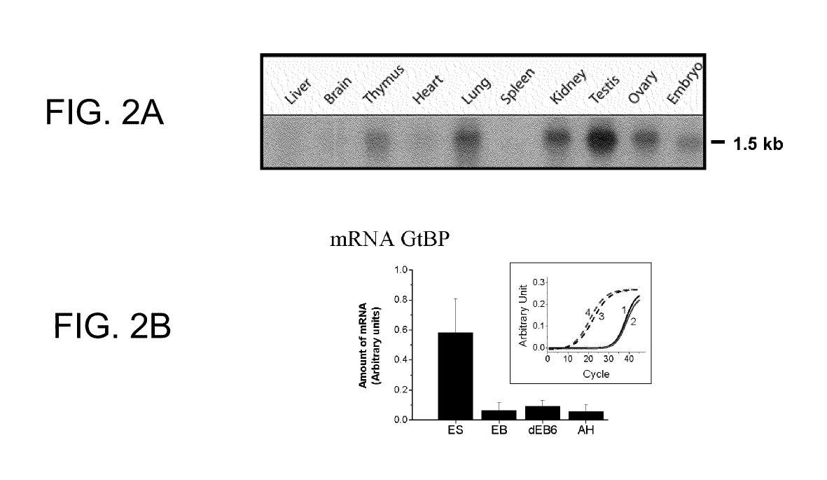

GTAP, a novel binding partner for Galactosyltransferase. GalT plays an important role in cell-to-cell contact and cell-matrix interactions. Regulation of this enzyme activity is crucial for many biological processes including egg-sperm binding during fertilization, early development and cell migration. In order to search for GalT associated proteins during stem cell development, we established a two hybrid yeast systems using the cytoplasmic domain of GalT consisting of 24 amino residues as bait to screen a 10-day old mouse embryonic cDNA library¹⁶. Eight putative positive clones were found and among them, a 500 bp cDNA clone called 26.11a showed high specificity (not shown). Further screening of a mouse embryonic λgt11 cDNA library, identified several overlapping clones, giving rise to a cDNA of ˜1.8 kb. In order to get the human homolog of GTAP, a human fetal heart library was used. The murine 26.11a cDNA show 98% homology to the human cDNA (FIG. 1). Because of its origin from the two hybrid yeast system and interaction to Galactosyltransferase (GalT) we named it GalT Binding Protein (GtBP), also referred to as GalT associated protein (GTAP). Northern blot hybridization using P³² labeled 5′ probe of 26.11a resulted in 1.7 kb band that showed up strong in reproductive organs, e.g., the testis and ovary. Weaker, yet positive, expression was found in kidney, lung, thymus and heart, but nearly negative in the liver, brain and spleen. (FIG. 2A.). Interestingly, mouse embryo was also positive for the 26.11a mRNA expression.

GTAP is Expressed During Early Development and is a New Member of Epidermal Differentiation Complex (EDC).

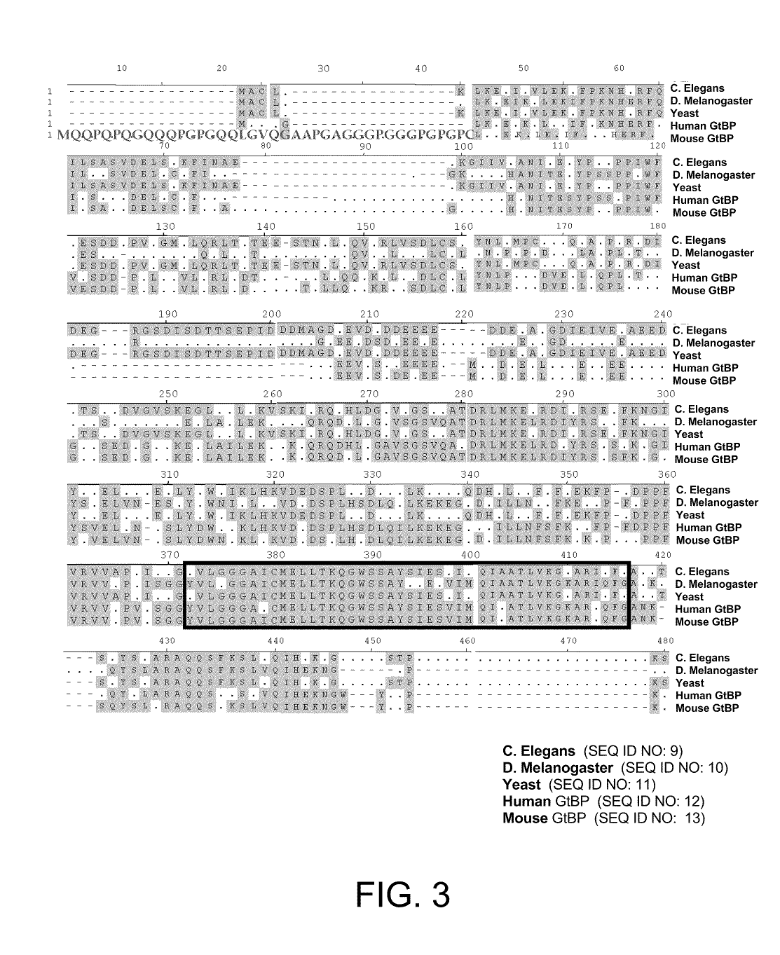

To see how GTAP mRNA changed during differentiation we used quantitative RT-PCR. As shown in FIG. 2B, there is a 10-fold decrease of GTAP mRNA during early differentiation. The amount of GTAP mRNA is low in adult tissues such heart. The cDNA sequence of GTAP was 95-100% similar to RIKEN cDNA located to mouse chromosome 3F1 (Genbank ID:AK009324). In human, this sequence mapped within a 2 MB area of chromosome 1q21 and was 50-70% similar to NICE5 protein (Genbank ID: AJ243666), a newly found member of the gene family called epidermal differentiation complex (EDC)⁴⁴. Furthermore, GTAP also showed about 50% protein sequence similarity to three other proteins of unknown function, murine NICE-5 like, a Drosophila Melanogaster gene EG:25E8 (accession no. AL009196), Caenorhabditis Elegans gene F25H2.8 (accession no. Z79754) and yeast. Interestingly, GTAP contains two specific domains, one glutamine and proline rich amino terminus and one carboxy terminal highly similar to ubiquitin conjugating enzyme domains (E2) (FIG. 3, enclosed in a box).

GTAP Binds to Cell Surface GalT and Regulates Cell-Matrix and Cell-Cell Adhesion During Early Embryonic Development.

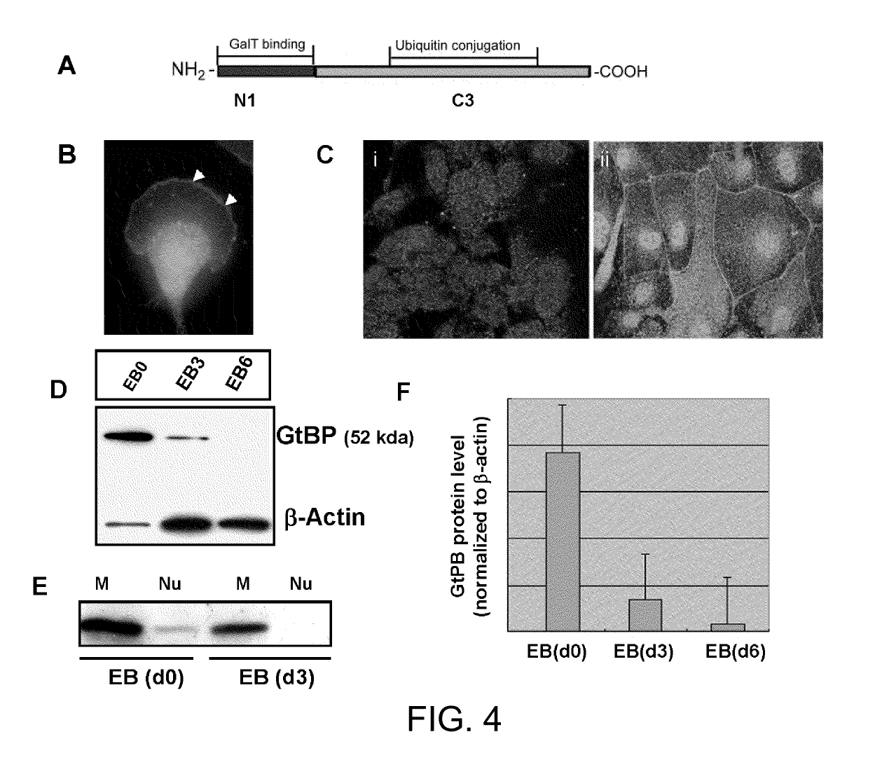

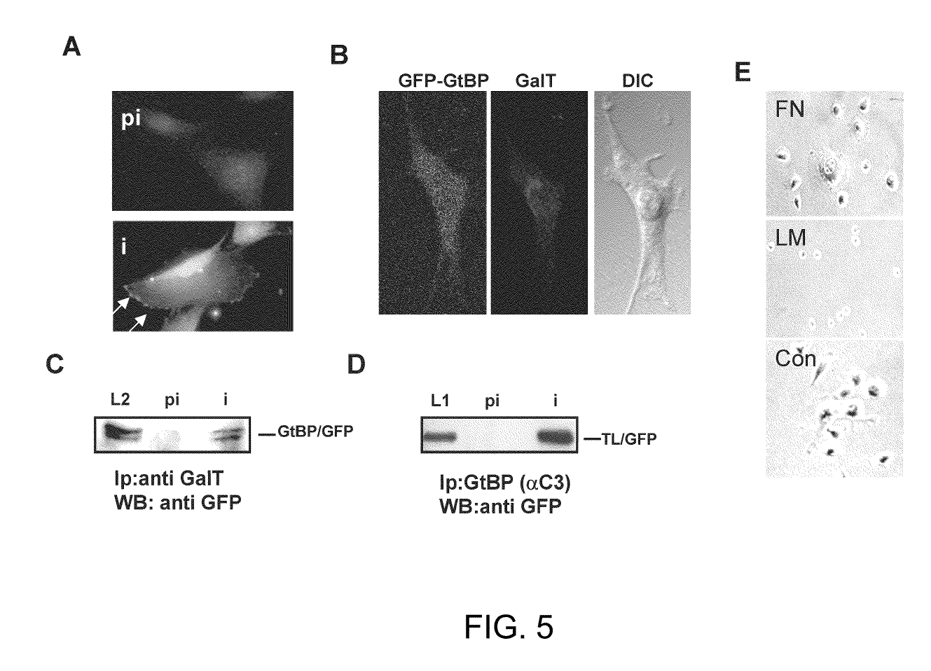

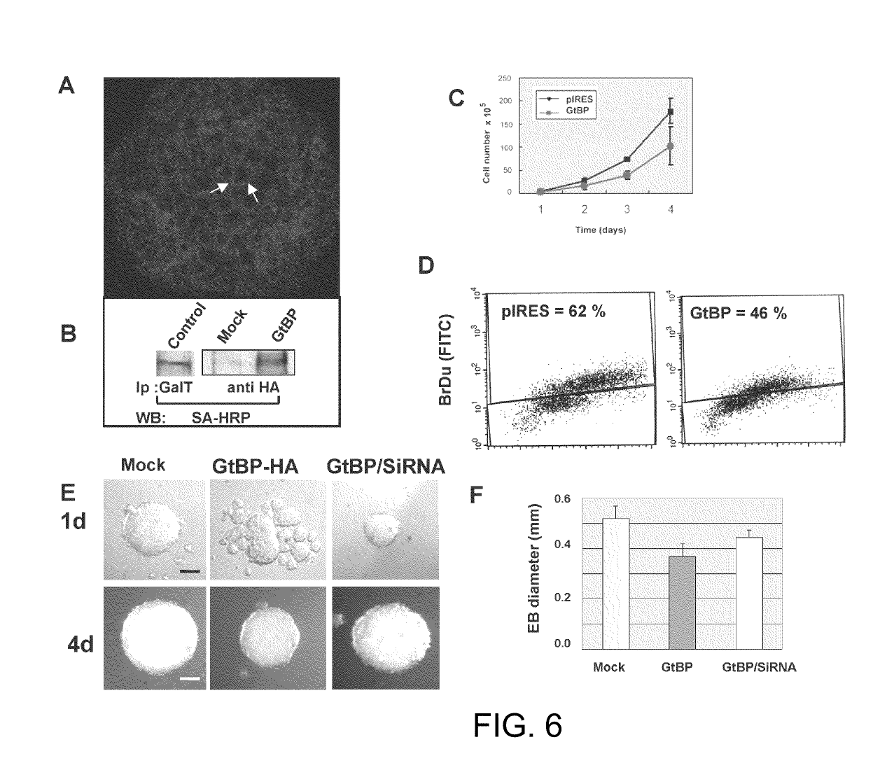

In order to determine tissue GTAP distribution and protein levels, antibodies were developed against a His-tagged amino terminal domain of GTAP (FIG. 4A, N1). In addition, antibodies were also made to detect the carboxy terminal end of GTAP using a GST fusion protein (FIG. 4A, anti C3). 3T3 NIH fibroblasts subjected to immunoflouresence using anti N1 clearly show that GTAP localizes to the lamelloopodia (FIG. 4B). Immunoprecipitation of lysates from undifferentiated cells with anti N1 and subsequent western blot with anti C3 of the protein resulted in a 60 kDa protein in embryonic stem cells (ESC), testis, 3T3 embryonic fibroblast. To characterize intracellular distribution of GTAP in embryonic cells, we performed immunofluorescence assays on differentiated EB (dEB day 1) with antibodies against N1. FIG. 4C show that GTAP localizes to intracellular contacts. The protein expression of GTAP decreases from undifferentiated ESC to a low level in late EBs (dEB6) (FIG. 4D, F). The decrease of GTAP during differentiation is not dependent on nuclear accumulation (FIG. 4E). To see if overexpression of GTAP could lead to aberration of proteins involved in stem cell growth, adhesion and differentiation, we constructed a plasmid containing cDNA coding for a fusion protein (GFP-GTAP) containing both green fluorescent protein and GTAP peptide sequences and transfected into 3T3 embryonic fibroblasts. As seen in (FIG. 5A) GFP-GTAP localized to the cytosol, philopodia, as well to the nucleus. To exclude the possibility that GTAP also binds to the Golgi form of GalT, we costained cells with both GalT and GFP antibodies. Confocal image indicated no co localization of GTAP and Golgi form of GalT (FIG. 5B). We found that antibodies to the catalytic domain of GalT could immunoprecipitate GFP-GTAP from lysates of GTAP cDNA transfected 3T3 cells (FIG. 5C). Inversely, a truncated form of GalT with its catalytic domain replaced by GFP (GFP-TL could be co-precipitated with antibodies against GTAP (FIG. 5D). Thus, GTAP acts as a GalT binding protein or GalT associated protein in embryonic fibroblasts. We next analyzed whether GTAP over expression affected GalT-related biological activities, such as cell-to-matrix binding. 3T3 NIH cells stably transfected with GFP-GTAP cDNA or with only GFP cDNA were plated on cell culture dishes coated with fibronectin or laminin. During 4-hours incubation, cells containing only GFP cDNA, settled down and spread out normally on laminin. To the contrary, cells over expressing GTAP-GFP lost the capability of spreading on laminin (FIG. 5E). This effect was laminin specific since transfected cells showed no effect on fibronectin. ESC ectopically expressing GTAP, have a similar dominant negative effect on cell adhesion on laminin as compared to embryonic fibroblast. The ectopically expressed GTAP localizes to intracellular contacts and binds to GalT (FIG. 6A, B). Growth curve analysis showed that there was a delay in the growth of cells expressing GTAP compared to control (FIG. 6C). Not surprisingly, FACS showed that cells ectopically expressing GTAP incorporated less BrDU than mock transfected cells (FIG. 6D). Furthermore, the growth of embryonic cells was stunted, forming smaller and less compact embryonic bodies (FIG. 6E, F). No effect was seen in GTAP knocked-down cells.

GTAP Regulate Cell Surface GalT and Cadherin/Catenin by Ubiquitination

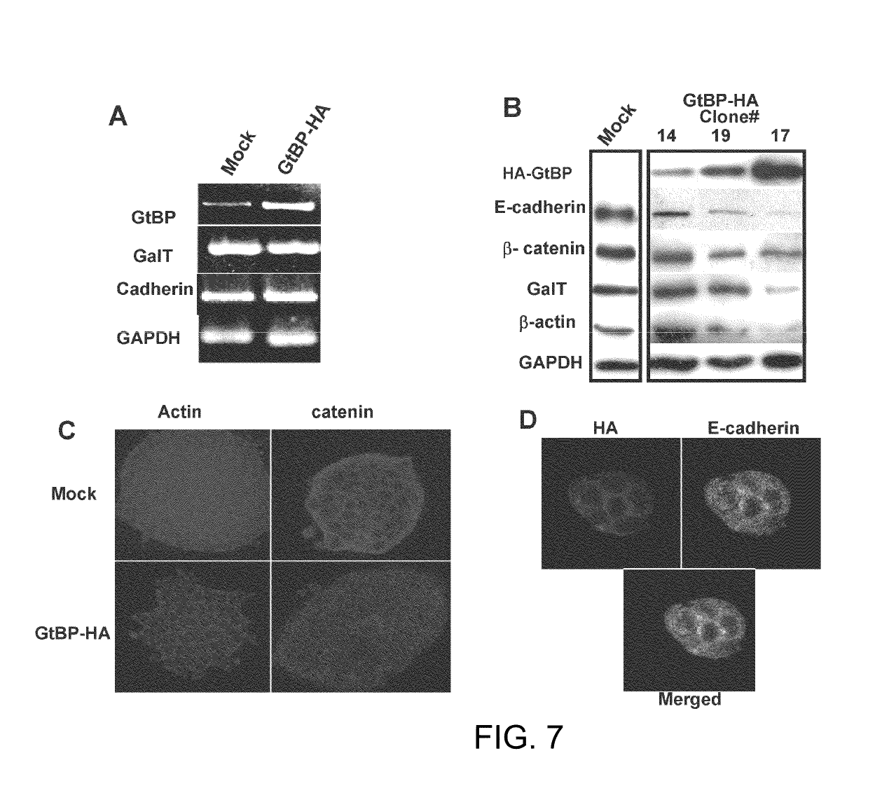

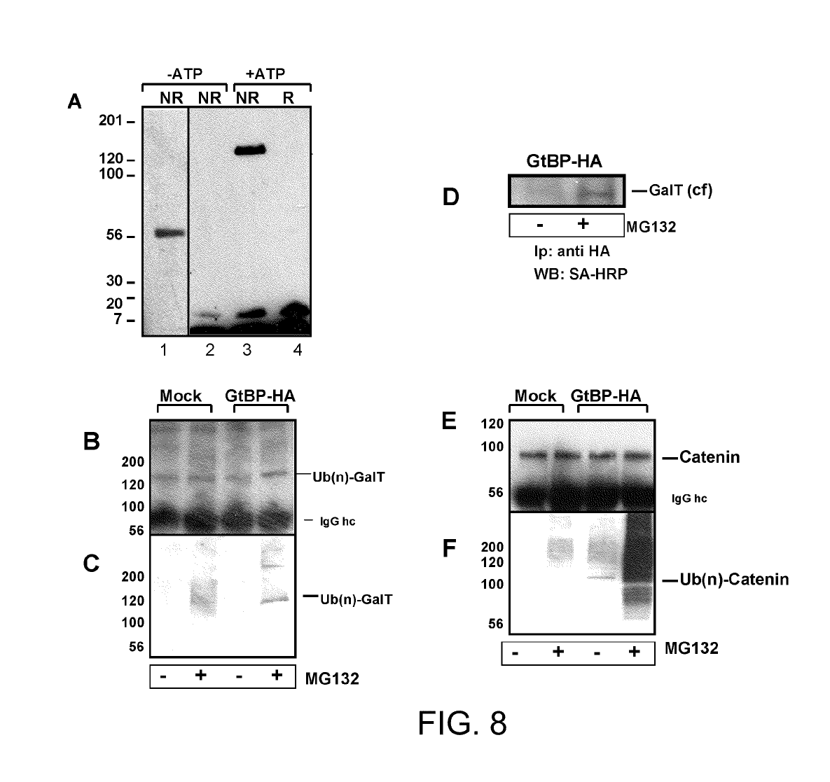

The effect of GTAP in cell adhesion and cell-cell interaction encouraged us to analyze GalT and E-cadherin protein level. FIG. 7B. Show western blot of lysates from stably transfected ESC. The protein levels of GalT, E-cadherin, catenin were significantly attenuated with increased level of the expressed GTAP transgene. This was not an effect of reduced expression since RT-PCR shows a constant level of mRNA for both proteins (FIG. 7A) Also beta-actin decreased. Immunofluorescence showed a reduced level of catenin (FIG. 7C). Furthermore, the ectopically expressed GTAP co-localize with cadherin (FIG. 7D). Since the carboxy terminal region of GTAP contains sequences that are homologous to the active domain of ubiquitin conjugating enzymes (Ubc's) we wanted to know if GTAP is able to form thiolester bonds to ubiquitin. Using His tagged GTAP we applied an in vitro ubiquination. FIG. 8A shows that His-GTAP is ubiquinylated in the presence of 1 mM ATP, migrating as a protein of <200 kDa. In the presence of DTT the ubiquitinylated products disappeared (FIG. 8A). These results suggest that the GTAP contains an active domain of ubiquitin conjugating enzymes. We further investigated a potential role for GTAP dependent ubiquitination of GalT and catenin. Cells were pretreated with either DMSO or MG132, an inhibitor of proteosome activity, lysed and subjected to immunoprecipitation using antibodies against GalT or catenin. The immunoprecipitates were analyzed for ubiquitination using antibodies against ubiquitin. As seen in FIGS. 8B and C, ubiquitinylated GalT accumulates in GTAP-HA expressing cells but not in mock transfected or in GTAP knock-down cells (not shown). Since GalT exists in both a Golgi form as well as in a membrane form, cell surface proteins were biotinylated in the presence of MG132, using sulpho-NHS-biotin, a non permeable derivative of biotin. The samples then were subjected to immunoprecipitation and western using strepavidin conjugated to horse radish peroxidase. Cells overexpressing GTAP and subjected to MG132, have an increased level of precipitable and biotinylated GalT compared to nontreated cells (FIG. 8D). Surprisingly GTAP had no effect on immunoprecipitated GalT in an in vitro ubiquitin system. Similarly, beta catenin was ubiquitinylated in a GTAP and proteosome-dependent way (FIG. 8E, F). These results together suggests that GTAP act as a new member of the ubiquitin degradation pathway regulating cell-cell contact during early development involving GalT and E-Cadherin.

GTAP Regulates Ubiquitination of the Cell Cyclin-Dependent Kinase Inhibitor p57^{Kip2}

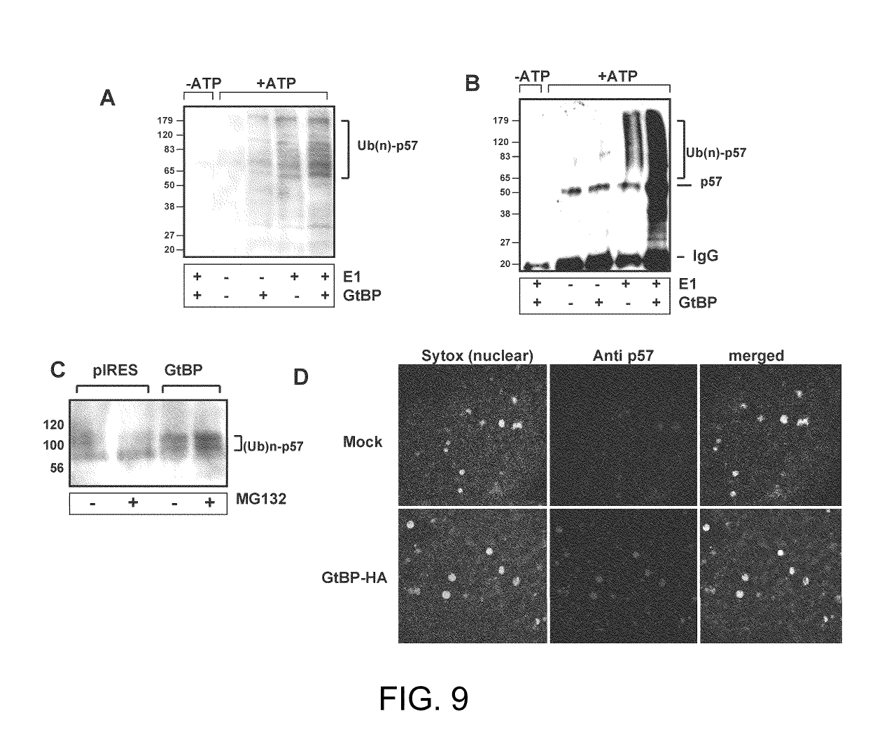

Cyclin-dependent kinase inhibitory proteins (CKIs) are negative regulators of the cell cycle. Of all CKIs, p57^{Kip2} plays an essential role in embryonic development. It has been shown earlier that p57 localizes to the nucleus in somatic cells, but less abundant in highly proliferative stem cell lines⁴⁵. Since overexpression of GTAP had a growth inhibitory effect on stem cells we first analyzed p57^{Kip2} in vitro ubiquitination. As seen in FIGS. 9A and B, GTAP increased the ubiquitination of p57 only in the presence of E1. In vivo, the GTAP ubiquinated forms of p57 accumulate in MG132 treated cells (FIG. 9C). Interestingly, more p57 localizes to the nucleus in GTAP transfected cells than in control (FIG. 9D). These results together suggest a regulatory function for GTAP in the ubiquitination and subsequent translocation of p57 to nucleolus.

Inhibitors of Protein Ubiquitination or GTAP Gene Expression

Polypeptides are synthesized in bacteria, yeast or mammalian cells by using recombinant DNA techniques with full-length and truncated GTAP cDNA. In modified or non-modified form, these polypeptides are used as regulators of ubiquitination by inhibiting or activating GTAP, dependent upon the modification under oxidation, acetylation, glycosylation or aldehyding. Ubiquitination of one or more cellular protein associated with cell adhesion, migration, proliferation, differentiation, cell-to-cell interaction, or any combination of those, may be altered by increasing or decreasing expression of GTAP by the cell. As a result, GTAP-mediated ubiquitination of one or more protein is respectively increased or decreased. The GTAP polypeptides are useful for making antibodies to GTAP, as well.

Polynucleotides are generated from GTAP cDNA sequences and used as the templates for production of small interference RNA. In addition, anti-GTAP antibodies, both monoclonal and polyclonal, may be generated. The polynucleotides may be used for altering survival, growth, adhesion or differentiation of a stem cell, a non-stem cell or a cancer cell by exposing the cell to one or more of the GTAP polynucleotides, which inhibit GTAP mediated protein ubiquitination or inhibit GTAP gene expression.

Discussion

During development and differentiation of stem cells, the surrounding extra cellular matrix and cell-cell interaction are of utmost importance for guidance of progenitor cells and for proper cell lineage commitment⁴⁶. Furthermore, signal transduction pathways controlling cell fate rely on a variety of carbohydrate-based modifications, including glycosylation of cell surface and extracellular matrix. There are a huge variety of cell surface receptors important for cell behavior, differentiation and cell survival. Cell-cell and cell matrix interactions deliver signals from the extracellular environment to the cell and vice versa. Laminin is one of the first extra cellular matrix proteins to be expressed in two to four-cell stage mouse embryos and is the major component of the extra cellular matrix of all basal lamina in vertebrates. One enzyme that has recently been implicated as a laminin receptor is β1,4-galactosyltransferase (GalT)^{12,47}. It has two isoforms due to differential translation, a short form located in the Golgi complex and a long form that has been shown to serve as a lectin-like cell surface receptor by virtue of its ability to interact with specific glycoside residues displayed on extracellular glycoproteins¹². Cell surface GalT is important for the regulation of intercellular adhesion between embryonic carcinoma cells (EC) and during late morula compaction in the preimplantation embryo⁴⁸. E-cadherin, which facilitates intercellular adhesions by homophilic binding, and cell surface GalT which binds terminal N-acetylglucosamin residues on consociated glycoprotein substrates on adjacent cell surfaces.

Through the yeast two-hybrid screen we successfully cloned a new protein called GalT binding protein (GtBP), also referred to as GalT associated protein (GTAP), from an embryonic cDNA library using the cytoplasmic domain of GalT as bait. The cDNA sequence was found to have 98% homology to human GTAP cDNA isolated from human fetal heart cDNA library (FIG. 1). Northern blot showed that GTAP is highly expressed in proliferative organs such as testis and ovary and in embryo (FIG. 2A). This spurred us to look for GTAP message in embryonic stem cells. Interestingly, undifferentiated mouse stem cells showed high GTAP mRNA level that decreased drastically during embryonic body (EB) formation. In adult tissues such as heart the level of GTAP mRNA was low (FIG. 2B)

Using NCBI blast search we found that GTAP cDNA sequence was 95-100% similar to RIKEN cDNA located to mouse chromosome 3F1 (genbank ID:AK009324). In human, this sequence mapped within a 2 MB area of chromosome 1q21 and was 50-70% similar to NICE5 protein (Genbank ID AJ243666), a newly found member of a gene family called the epidermal differentiation complex (EDC). Furthermore, GTAP also showed about 50% protein sequence similarity to two other proteins of unknown function, one deduced from Drosophila Melanogaster gene EG:25E8 (accession no. AL009196), a yeast ubiquitin conjugating enzyme and Caenorhabditis Elegans gene F25H2.8 (accession no. Z79754) (FIG. 3). The ORF of GTAP reveals similarity to two interesting domains; first, a proline-rich region located in the amino terminal end. This kind of sequences are often seen in many small proline-rich proteins (SPRPs) and resembles highly conserved glutamine repetitive sequences thought to be crucial for the regulation of cell proliferation and differentiation. Secondly, a structurally conserved region of ubiquitin conjugating enzymes (E2) located in the carboxy terminal end of GTAP (C3)

Using antibodies against the amino terminal region of GTAP and the carboxy terminal region (FIG. 4A, N1 and C3) we were able to immunoprecipitate a protein of ˜55 kDa from both testis and 3T3 cell lysates This binding was specific since a protein of a molecular weight ˜60 kDa, representing GalT, were coprecipitated with GTAP in wild type but not in GalT-null testis. GTAP localize to the cytosol, nucleus as well as lamellopodia in embryonic fibroblast (FIG. 4B). There was no staining of GTAP in the Golgi which has been shown for earlier GalT. Similarly, during the early stages of differentiation (0-3 days) of embryonic stem cells, GTAP localized to intracellular junctions (FIG. 4C). Both mRNA and protein levels of GTAP declined when undifferentiated stem cells formed embryoid bodies composed of a variety of functionally specialized cells seen in adult tissues or organs, including cardiovascular cells, nerve cells, and blood cells (FIG. 4D).

Immunofluorescent scanning confocal microscopy demonstrated that unlike other ubiquitin-carrying enzymes, GTAP seems bound to cell membrane and located in the nuclei. The unique localization of GTAP promoted us to analyze the biological effects on cells ectopically expressing GTAP. In embryonic fibroblasts, increased expression of GTAP induced a decrease in cell adhesion on laminin but not fibronectin (FIG. 5E). To ensure that the reduced cell adhesion is due to GTAP-mediated membrane protein ubiquitination, a cell line expressing a GTAP-GFP (green fluorescence protein) fusion protein. The ectopically expressed GTAP was immunoprecipitated as a protein doublet using antibodies against the extracellular domain of GalT. Since the cDNA corresponding to GFP was located upstream of from the GTAP, the protein doublet suggests posttranslational modification of GalT, such as ubiquitination or phosphorylation since the doublet was also detected in immunoprecipitation. Since many different isomers of GalT have been identified during recent years and hence could be a problem in the interpretation of the specificity of the interaction, a truncated version of cell surface GalT was made where the catalytic domain was exchanged for GFP, GFP-TL^{49,50}. As expected, the ectopically expressed GFP-TL was coprecipitated with antibodies against the carboxyterminus of GTAP (FIG. 5D)

Laminin constitutes an important matrix protein for not only for cell spreading and migration but also for propagation and differentiation of embryonic stem cells. Interestingly, cell surface GalT has been detected as early as in embryonic carcinoma as an important regulator of cell growth, cell-cell contact and laminin synthesis. Similar to the effect on embryonic fibroblasts, undifferentiated stem cells ectopically expressing GTAP fused to hemaglutinin, could not adhere properly to extracellular laminin.

Because ectopically expressed GTAP associated with GalT in intracellular contacts of undifferentiated cells (FIGS. 6A and B). Interestingly, as seen in FIG. 6C-D, also the growth of undifferentiated and formation embryonic bodies (EB's) during the first stage of embryonic stem cell differentiation were attenuated. In contrast, mock transfected and stem cells subjected to siRNA technology, were still able to form EB's (FIG. 6E). As seen in FIG. 7B there was a correlation between GTAP expression and the loss of GalT, cadherin/catenin and β-actin. This loss, however, was not due to a decrease in transcription since GTAP over expression showed no effect on mRNA level of either GalT or E-cadherin. Since the amino terminal end of GTAP had a homologous domain to ubiquitin conjugating like enzymes, we first analyzed the ability of GTAP to form thiolester bonds to ubiquitin. We found that GTAP could bind ubiquitin in an ATP and thiol ester dependent manner in an in vitro system (FIG. 8A). A protein complex of a molecular weight of >200 kDa was precipitated with nickel beads only in the presence of ATP. Recent experiments have indicated the importance of the ubiquitin pathway in proliferation and differentiation of dentritic cells, epidermal as well as ectodermal cells during development. Furthermore, a recent report shows that a major burst of ubiquitin-dependent proteolysis occurs in the trophoblast of mammalian peri-implantation embryos. This event may be important for the success of blastocyst hatching, differentiation of embryonic stem cells into soma and germ line, and/or implantation in both naturally conceived and reconstructed mammalian embryos⁵¹.

Apart from the effect of ectopically expressed GTAP, we were not able to see an effect on GTAP knock-down cells, which is in agreement with an earlier report showing that disruption of the gene encoding for UbcM4, another ubiquitin conjugating enzyme found in stem cells, had no obvious effect on proliferation and in vitro differentiation of mouse embryonic stem cells⁵². If a GTAP dependent degradation pathway for GalT exists, we would expect the protein level of the receptor to decrease in cells over expressing GTAP compared to control cells. This was, in fact, the case since cells treated with a protesome accumulated ubiquitinylated GalT (FIG. 8B-C). Because of the relatively low abundance of the cell surface form of GalT, this result is hard to interpret. Immunoprecipitation experiments using lysates from cells subjected to cell surface biotinylation showed that GalT accumulated in a GTAP and proteosome dependent way (FIG. 8D). The decrease of cell surface GalT could be accomplished by the binding of a GTAP-Ubiquitin complex to GalT cytoplasmic domain inducing internalization and degradation through the endocytotic pathway. It is interesting to note that the 24-amino acid cytoplasmic domain of the cell surface form of GalT contains two distal lysines constituting potential targets for ubiquination⁴. Interestingly, replacing either the serine or threonine residues on the cytoplasmic domain with aspartic acid reduced the surface expression and function suggesting that phosphorylation could potentially regulate GalT function on the cell surface⁴⁹. Consistent with this result, phosphorylation of the cytoplasmic domains in two GTP protein-coupled signal transducing receptors, α-factor and α-factor, implicated in the pheromone response regulate the association to two ubiquitin conjugating enzymes Ubc 4p and Ubc5p for degradation⁵³. Compared to mock transfected cells, the level of cell surface GalT was efficiently abolished in the presence of ectopically expressed GTAP. Moreover, GTAP dependent ubiquitination of GalT was only detected in vivo in the presence of the proteosome inhibitor MG132 and not in vitro. These results suggest that another component in the cell not bound to GalT is needed for efficient ubiquitination of GalT. GTAP/GalT together could act as an E3 ligase complex that potentially could recognize and ubiquinylate other proteins in the vicinity of cell surface GalT or other important signal transduction proteins. E-cadherin has been shown to be a substrate for cell surface GalT, suggesting that it may participate in GalT-specific adhesions and growth⁵⁴. Consistent with this data, the in vivo but not in vitro assay showed that cadherin/catenin and associated proteins were efficiently ubiquitinylated only in the presence of MG132. Interestingly, interaction between GalT and E-cadherin has been shown to exhibit characteristic changes during retinoic acid induced F9 cell differentiation.

Concomitant with the decrease in cell adhesion and cell-cell interaction, cells overexpressing GTAP tended to grow much slower than mock transfected cells. This could be due to alteration of the G1-S phase transition since GTAP transfected cells incorporated 20% less BrDu compared to mock transfected cells (46 compared to 62%). The abundance of the cyclin-dependent kinase (CDK) inhibitor p57^{kip2}, an important regulator of cell cycle progression, has been suggested to be controlled by the ubiquitin-proteosome pathway through the Skp1/Cull/F-box complex (SCF) important in the G1-S progression. We propose that GtPB is a regulator of p57 ubiquitination. In support of this hypothesis, ubiquitination of p57 was increased in vitro dependent on GTAP. Also, lysates from MG132 treated cells ectopically expressing GTAP showed an increased level of ubiquitinylated p57. However, as seen in western blot analysis from lysates of stable cell lines, there was no apparent change in p57 protein level between mock and cells overexpressing GTAP. Interestingly, there were more p57 reactive nuclei in transfectants. It was reported that an S-phase kinase associated protein 2 (Skp2) is necessary to promote ubiquitin-mediated degradation through the SCF complex^{55,56}. It is possible that an increase of plasma membrane associated GalT leads to an accumulation of bound GTAP. This may lead to downregulation of Skp2 activity, thereby stabilizing the half-life of p57 and subsequent slower G1 to S phase transition. Considering GTAP's interaction to the cell surface form of GalT and its cell growth regulating properties it is interesting to note that cell surface GalT has been shown to be upregulated in metastasis. Maybe more intriguing, the expression of GalT was shown to be cell cycle specific, with the cell surface and intracellular GalT pools displaying independent expression patterns. Stably transfected cell lines with reduced levels of cytoskeletally associated surface GalT grew faster than control cells, whereas cell lines that over-expressed surface GalT grew slower than controls.

### REFERENCES

The following publications are cited by number in the foregoing text.
1. Odorico J S, Kaufman D S, Thomson J A. Multilineage differentiation from human embryonic stem cell lines. Stem Cells. 2001; 19:193-204.
2. Pittenger M F, Mackay A M, Beck S C, Jaiswal R K, Douglas R, Mosca J D, Moorman M A, Simonetti D W, Craig S, Marshak D R. Multilineage potential of adult human mesenchymal stem cells. Science. 1999; 284:143-7.
3. Ramakrishnan S, Kothari S S, Bahl V K. Stem cells and myocardial regeneration. Indian Heart J. 2003; 55:119-24.
4. Rechsteiner M C. Ubiquitin-mediated proteolysis: an ideal pathway for systems biology analysis. Adv Exp Med Biol. 2004; 547:49-59.
5. Passmore L A, Barford D. Getting into position: the catalytic mechanisms of protein ubiquitylation. Biochem J. 2004; 379:513-25.
6. Glickman M H. Getting in and out of the proteasome. Semin Cell Dev Biol. 2000; 11:149-58.
7. Hicke L, Dunn R. Regulation of membrane protein transport by ubiquitin and ubiquitin-binding proteins. Annu Rev Cell Dev Biol. 2003; 19:141-72.
8. Gagneux P, Varki A. Evolutionary considerations in relating oligosaccharide diversity to biological function. Glycobiology. 1999; 9:747-55.
9. Varki A. “Unusual” modifications and variations of vertebrate oligosaccharides: are we missing the flowers for the trees? Glycobiology. 1996; 6:707-10.
10. Manzi A E, Norgard-Sumnicht K, Argade S, Marth J D, van Halbeek H, Varki A. Exploring the glycan repertoire of genetically modified mice by isolation and profiling of the major glycan classes and nano-NMR analysis of glycan mixtures. Glycobiology. 2000; 10:669-89.
11. Varki A. Factors controlling the glycosylation potential of the Golgi apparatus. Trends Cell Biol. 1998; 8:34-40.
12. Shur B D, Evans S, Lu Q. Cell surface galactosyltransferase: current issues. Glycoconj J. 1998; 15:537-48.
13. Russo R N N, Shaper N L, Shaper J H. Bovine beta 1-4-galactosyltransferase: two sets of mRNA transcripts encode two forms of the protein with different amino-terminal domains. In vitro translation experiments demonstrate that both the short and the long forms of the enzyme are type II membrane-bound glycoproteins. J Biol Chem. 1990; 265:3324-31.
14. Russo R N, Shaper N L, Taatjes D J, Shaper J H. Beta 1,4-galactosyltransferase: a short NH2-terminal fragment that includes the cytoplasmic and transmembrane domain is sufficient for Golgi retention. J Biol Chem. 1992; 267:9241-7.
15. Wassler M J, Shur B D. Clustering of cell surface (beta)1,4-galactosyltransferase I induces transient tyrosine phosphorylation of focal adhesion kinase and loss of stress fibers. J Cell Sci. 2000; 113 Pt 2:237-45.
16. Wassler M J, Foote C I, Gelman I H, Shur B D. Functional interaction between the SSeCKS scaffolding protein and the cytoplasmic domain of beta1,4-galactosyltransferase. J Cell Sci. 2001; 114:2291-300.
17. Lu Q, Hasty P, Shur B D. Targeted mutation in beta-1,4-galactosyltransferase leads to pituitary insufficiency and neonatal lethality. Dev Biol. 1997; 181:257-67.
18. Koch A W, Manzur K L, Shan W. Structure-based models of cadherin-mediated cell adhesion: the evolution continues. Cell Mol Life Sci. 2004; 61:1884-95.
19. Gooding J M, Yap K L, Ikura M. The cadherin-catenin complex as a focal point of cell adhesion and signalling: new insights from three-dimensional structures. Bioessays. 2004; 26:497-511.
20. Ham C, Levkau B, Raines E W, Herren B. ADAM15 is an adherens junction molecule whose surface expression can be driven by VE-cadherin. Exp Cell Res. 2002; 279:239-47.
21. Steinhusen U, Weiske J, Badock V, Tauber R, Bommert K, Huber O. Cleavage and shedding of E-cadherin after induction of apoptosis. J Biol Chem. 2001; 276:4972-80.
22. Larue L, Ohsugi M, Hirchenhain J, Kemler R. E-cadherin null mutant embryos fail to form a trophectoderm epithelium. Proc Natl Acad Sci USA. 1994; 91:8263-7.
23. Larue L, Antos C, Butz S, Huber O, Delmas V, Dominis M, Kemler R. A role for cadherins in tissue formation. Development. 1996; 122:3185-94.
24. Rosenberg P, Esni F, Sjodin A, Lame L, Carlsson L, Gullberg D, Takeichi M, Kemler R, Semb H. A potential role of R-cadherin in striated muscle formation. Dev Biol. 1997; 187:55-70.
25. Dahl U, Sjodin A, Lame L, Radice G L, Cajander S, Takeichi M, Kemler R, Semb H. Genetic dissection of cadherin function during nephrogenesis. Mol Cell Biol. 2002; 22:1474-87.
26. Haegel H, Lame L, Ohsugi M, Fedorov L, Herrenknecht K, Kemler R. Lack of beta-catenin affects mouse development at gastrulation. Development. 1995; 121:3529-37.
27. Novak A, Dedhar S. Signaling through beta-catenin and Lef/Tcf. Cell Mol Life Sci. 1999; 56:523-37.
28. Hurlstone A F, Haramis A P, Wienholds E, Begthel H, Korving J, Van Eeden F, Cuppen E, Zivkovic D, Plasterk R H, Clevers H. The Wnt/beta-catenin pathway regulates cardiac valve formation. Nature. 2003; 425:633-7.
29. Nakamura T, Sano M, Songyang Z, Schneider M D. A Wnt- and beta-catenin-dependent pathway for mammalian cardiac myogenesis. Proc Natl Acad Sci USA. 2003; 100:5834-9.
30. Kioussi C, Briata P, Baek S H, Rose D W, Hamblet N S, Herman T, Ohgi K A, Lin C, Gleiberman A, Wang J, Brault V, Ruiz-Lozano P, Nguyen H D, Kemler R, Glass C K, Wynshaw-Boris A, Rosenfeld M G. Identification of a Wnt/Dv1/beta-Catenin-->Pitx2 pathway mediating cell-type-specific proliferation during development. Cell. 2002; 111:673-85.
31. Baek S H, Kioussi C, Briata P, Wang D, Nguyen H D, Ohgi K A, Glass C K, Wynshaw-Boris A, Rose D W, Rosenfeld M G. Regulated subset of G1 growth-control genes in response to derepression by the Wnt pathway. Proc Natl Acad Sci USA. 2003; 100:3245-50.
32. Zhang P, Wong C, DePinho R A, Harper J W, Elledge S J. Cooperation between the Cdk inhibitors p27(KIP1) and p57(KIP2) in the control of tissue growth and development. Genes Dev. 1998; 12:3162-7.
33. Dyer M A, Cepko C L. p27Kip1 and p57Kip2 regulate proliferation in distinct retinal progenitor cell populations. J Neurosci. 2001; 21:4259-71.
34. Lee M H, Reynisdottir I, Massague J. Cloning of p57KIP2, a cyclin-dependent kinase inhibitor with unique domain structure and tissue distribution. Genes Dev. 1995; 9:639-49.
35. Reynaud E G, Leibovitch M P, Tintignac L A, Pelpel K, Guillier M, Leibovitch S A. Stabilization of MyoD by direct binding to p57(Kip2). J Biol Chem. 2000; 275:18767-76.
36. Reynaud E G, Pelpel K, Guillier M, Leibovitch M P, Leibovitch S A. p57(Kip2) stabilizes the MyoD protein by inhibiting cyclin E-Cdk2 kinase activity in growing myoblasts. Mol Cell Biol. 1999; 19:7621-9.
37. Grandjean V, Smith J, Schofield P N, Ferguson-Smith A C. Increased IGF-II protein affects p57kip2 expression in vivo and in vitro: implications for Beckwith-Wiedemann syndrome. Proc Natl Acad Sci USA. 2000; 97:5279-84.
38. Marenholz I, Volz A, Ziegler A, Davies A, Ragoussis I, Korge B P, Mischke D. Genetic analysis of the epidermal differentiation complex (EDC) on human chromosome 1q21: chromosomal orientation, new markers, and a 6-Mb YAC contig. Genomics. 1996; 37:295-302.
39. Causier B. Studying the interactome with the yeast two-hybrid system and mass spectrometry. Mass Spectrom Rev. 2004; 23:350-67.
40. Thaminy S, Miller J, Stagljar I. The split-ubiquitin membrane-based yeast two-hybrid system. Methods Mol Biol. 2004; 261:297-312.
41. Nguyen T T, Hinton D A, Shur B D. Expressing murine beta 1,4-galactosyltransferase in HeLa cells produces a cell surface galactosyltransferase-dependent phenotype. J Biol Chem. 1994; 269:28000-9.
42. Tengowski M W, Wassler M J, Shur B D, Schatten G. Subcellular localization of beta1,4-galactosyltransferase on bull sperm and its function during sperm-egg interactions. Mol Reprod Dev. 2001; 58:236-44.
43. Nixon B, Lu Q, Wassler M J, Foote C I, Ensslin M A, Shur B D. Galactosyltransferase function during mammalian fertilization. Cells Tissues Organs. 2001; 168:46-57.
44. Marenholz I, Zirra M, Fischer D F, Backendorf C, Ziegler A, Mischke D. Identification of human epidermal differentiation complex (EDC)-encoded genes by subtractive hybridization of entire YACs to a gridded keratinocyte cDNA library. Genome Res. 2001; 11:341-55.
45. Hiromura K, Haseley L A, Zhang P, Monkawa T, Durvasula R, Petermann A T, Alpers C E, Mundel P, Shankland S J. Podocyte expression of the CDK-inhibitor p57 during development and disease. Kidney Int. 2001; 60:2235-46.
46. Lebkowski J S, Gold J, Xu C, Funk W, Chiu C P, Carpenter M K. Human embryonic stem cells: culture, differentiation, and genetic modification for regenerative medicine applications. Cancer J. 2001; 7 Suppl 2:S83-93.
47. Rodeheffer C, Shur B D. Targeted mutations in beta-1,4-galactosyltransferase I reveal its multiple cellular functions. Biochim Biophys Acta. 2002; 1573:258-70.
48. Bayna E M, Shaper J H, Shur B D. Temporally specific involvement of cell surface beta-1.4 galactosyltransferase during mouse embryo morula compaction. Cell. 1988; 53:145-57.
49. Hathaway H J, Evans S C, Dubois D H, Foote C I, Elder B H, Shur B D. Mutational analysis of the cytoplasmic domain of beta-1,4-galactosyltransferase I: influence of phosphorylation on cell surface expression. J Cell Sci. 2003; 116:4319-30.
50. Hathaway H J, Shur B D. Mammary gland morphogenesis is inhibited in transgenic mice that overexpress cell surface beta-1,4-galactosyltransferase. Development. 1996; 122:2859-72.
51. Sutovsky P, Motlik J, Neuber E, Pavlok A, Schatten G, Palecek J, Hyttel P, Adebayo O T, Adwan K, Alberio R, Bagis H, Bataineh Z, Bjerregaard B, Bodo S, Bryja V, Carrington M, Couf M, de la Fuente R, Diblik J, Esner M, Forejt J, Fulka J, Jr., Geussova G, Gjorret J O, Libik M, Hampl A, Hassane M S, Houshmand M, Hozak P, Jezova M, Kania G, Kanka J, Kandil O M, Kishimoto T, Klima J, Kohoutek J, Kopska T, Kubelka M, Lapathitis G, Laurincik J, Lefevre B, Mihalik J, Novakova M, Oko R, Omelka R, Owiny D, Pachernik J, Pacholikova J, Peknicova J, Pesty A, Ponya Z, Preclikova H, Sloskova A, Svoboda P, Strejcek F, Toth S, Tepla O, Valdivia M, Vodicka P, Zudova D. Accumulation of the proteolytic marker peptide ubiquitin in the trophoblast of mammalian blastocysts. Cloning Stem Cells. 2001; 3:157-61.
52. Pring a E, Meier I, Muller U, Martinez-Noel G, Harbers K. Disruption of the gene encoding the ubiquitin-conjugating enzyme UbcM4 has no effect on proliferation and in vitro differentiation of mouse embryonic stem cells. Biochim Biophys Acta. 2000; 1494:75-82.
53. Hicke L. Gettin' down with ubiquitin: turning off cell-surface receptors, transporters and channels. Trends Cell Biol. 1999; 9:107-12.
54. Hinton D A, Evans S C, Shur B D. Altering the expression of cell surface beta 1,4-galactosyltransferase modulates cell growth. Exp Cell Res. 1995; 219:640-9.
55. Kamura T, Koepp D M, Conrad M N, Skowyra D, Moreland R J, Iliopoulos O, Lane W S, Kaelin W G, Jr., Elledge S J, Conaway R C, Harper J W, Conaway J W. Rbx1, a component of the VHL tumor suppressor complex and SCF ubiquitin ligase. Science. 1999; 284:657-61.
56. Skowyra D, Koepp D M, Kamura T, Conrad M N, Conaway R C, Conaway J W, Elledge S J, Harper J W. Reconstitution of G1 cyclin ubiquitination with complexes containing SCFGrr1 and Rbx1. Science. 1999; 284:662-5.

Without further elaboration, it is believed that one skilled in the art can, using the description herein, utilize the present invention to its fullest extent. The foregoing embodiments are to be construed as illustrative, and not as constraining the remainder of the disclosure in any way whatsoever. While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims. The disclosures of all patents, patent applications and publications cited herein are hereby incorporated herein by reference, to the extent that they provide exemplary, procedural or other details supplementary to those set forth herein.

What is claimed is:

1. A method of altering ubiquitination of at least one cellular protein of a stem cell, comprising increasing or decreasing expression of GalT binding protein (GtBP) by said stem cell, whereby GtBP-mediated ubiquitination of at least one cellular protein is correspondingly increased or decreased in said stem cell.

2. The method of claim 1, wherein said GtBP comprises SEQ ID NO: 12.

3. The method of claim 1, wherein said increasing or decreasing of GtBP expression comprises altering the levels of GtBP mRNA and protein in said stem cell.

4. The method of claim 3, wherein said altering the levels of GtBP mRNA and protein comprises exposing said stem cell to a GtBP protein, a GtBP fusion protein, polypeptide or active fragment thereof.

5. The method of claim 3, wherein altering the levels of GtBP mRNA and protein in said stem cell comprises exposing said stem cell to a polypeptide inhibitor of GtBP-mediated ubiquitination or an antibody that binds to GtBP, to decrease ubiquitination of said at least one cellular protein.

6. The method of claim 5, wherein said polypeptide inhibitor comprises a dominant-negative polypeptide analog of GtBP which lacks a functional domain of GtBP or lacks a cofactor binding site of GtBP.

7. The method of claim 3, wherein altering the levels of GtBP mRNA and proteins in said stem cell comprises exposing said stem cell to a polynucleotide inhibitor of GtBP gene expression to decrease ubiquitination of said at least one cellular protein.

8. The method of claim 5, wherein said polynucleotide inhibitor comprises a small double-strand interference RNA targeting to GtBP mRNA.

9. The method of claim 8, wherein said small double-strand interference RNA is encoded by a truncated portion of either SEQ ID NO.: 7 or SEQ ID NO.: 8.

10. The method of claim 1, wherein said at least one cellular protein comprises a membrane protein.

11. The method of claim 1, wherein said increasing expression of GtBP increases ubiquitination of a said protein associated with cell adhesion or cell-cell interaction causing a decrease in cell adhesion and/or cell-cell interaction of said stem cell, or said decreasing expression of GtBP decreases ubiquitination of said protein associated with cell adhesion or cell-cell interaction causing an increase in cell adhesion and/or cell-cell interaction of said stem cell.

12. The method of claim 1, wherein said increasing expression of GtBP correlates with a decrease in the amount of at least one cell surface protein, or said decreasing expression of GtBP correlates with an increase in the amount of at least one cell surface protein in said stem cell.

13. The method of claim 1, wherein said increasing expression of GtBP correlates with an increase in the level of GtBP-mediated ubiquitination of GalT in said stem cell, or said decreasing expression of GtBP correlates with a decrease in the level of GtBP-mediated ubiquitination of GalT in said stem cell.

14. The method of claim 13, wherein said increase in the level of GtBP-mediated ubiquitination of GalT decreases cell adhesion in said stem cell, or said decrease in the level of GtBP-mediated ubiquitination of GalT increases cell adhesion in said stem cell.

15. The method of claim 1, wherein said increase in the level of GtBP-mediated ubiquitination of GalT deters differentiation of said stem cell, or said decrease in the level of GtBP-mediated ubiquitination of GalT promotes differentiation of said stem cell.

16. The method of claim 15, wherein said increase in the level of GtBP-mediated ubiquitination of GalT increases in vitro survival of said stem cell.

17. The method of claim 15, wherein said increase in the level of GtBP-mediated ubiquitination of GalT increases cell growth.

18. The method of claim 15, wherein said increasing or decreasing expression of GtBP comprises introducing into said stem cell a recombinant plasmid expressing GtBP.

19. The method of claim 1, wherein, in said increasing or decreasing expression of GtBP comprises causing ectopic expression of GalT associated protein.

20. A method of indexing the pluripotency of a stem cell, the method comprising:
assessing the level of GtBP-mediated polyubiquitination of said stem cell; and
correlating said level of polyubiquitination with pluripotency, multipotency, oligopotency or monopotency of said stem cell for growth, survival and differentiation into a blood or somatic tissue or organ cell type.
